(19) United States
(12) Patent Application Publication
(10) Pub. No.: US 2026/0263002 A1
(43) Pub. Date: Sep. 10, 2026

(54) MEASURING MAXIMAL OXYGEN CONSUMPTION

(71) Applicant: Whoop, Inc., Boston, MA (US)

(72) Inventors: Maxwell Richard Fernandes Perozek, Cambridge, MA (US); Victoria Harrison Lee, Waltham, MA (US); Eric Robert Dougherty, Nashua, NH (US); David Mikal Presby, Bern (CH); Laura Ware, Waltham, MA (US); Godine Kok Yan Chan, Natick, MA (US); William Ricardo Sweeney, Brookline, MA (US)

(21) Appl. No.: 19/561,023

(22) Filed: Mar. 9, 2026

Related U.S. Application Data
(60) Provisional application No. 63/769,093, filed on Mar. 9, 2025.

(51) Int. Cl. A61B 5/00; A61B 5/0205; A61B 5/024; A61B 5/11; A61B 5/1455; G16H 40/67; G16H 50/30

(52) U.S. Cl. CPC A61B 5/4866; A61B 5/0205; A61B 5/7278; A61B 5/742; G16H 40/67; G16H 50/30; A61B 5/02438; A61B 5/1112; A61B 5/14551; A61B 5/4806

(57) ABSTRACT

A monitoring system supports numerous modes for calculating a periodic, e.g., daily, estimate of VO2 Max. A variety of strategies are used to select from among these modes based on the nature of supporting data that is available at the time that the VO2 Max estimate is calculated, and to transition in a data-driven way among these modes as higher-quality data sources become available or as these data sources become obsolete.

### RELATED APPLICATIONS

This application claims priority to U.S. Prov. App. No. 63/769,093 filed on Mar. 9, 2025, the entire content of which is hereby incorporated by reference.

This application is also related to International Patent App. No. PCT/US26/18336 filed on Mar. 9, 2026, which claims priority to U.S. Prov. App. No. 63/769,093 filed on Mar. 9, 2025, where the entire content of each of the foregoing is hereby incorporated by reference.

### BACKGROUND

A wearable physiological monitor may be used to track maximal oxygen consumption (VO2 Max) for a user. However, there remains a need for improved techniques to monitor VO2 Max on an ongoing basis without imposing strenuous physical protocols or requiring user attention.

### SUMMARY

A monitoring system supports numerous modes for calculating a periodic, e.g., daily, estimate of VO2 Max. A variety of strategies are used to select from among these modes based on the nature of supporting data that is available at the time that the VO2 Max estimate is calculated, and to transition in a data-driven way among these modes as higher-quality data sources become available or as these data sources become obsolete.

In one aspect, a method disclosed herein includes: providing a first model for estimating a maximal oxygen consumption for a user based on user data, the user data including physiological data acquired from a wearable physiological monitor worn by the user; receiving supplemental data relating to the maximal oxygen consumption; transitioning to a second model for estimating the maximal oxygen consumption based on the user data and the supplemental data; and calculating an estimated maximal oxygen consumption based on the second model.

The method may include displaying the estimated maximal oxygen consumption to the user. The method may include calculating the maximal oxygen consumption once per week based on an historical window of data for the user. The method may include calculating the maximal oxygen consumption once per day based on an historical window of data for the user. The first model may include a regression model. The user data may include photoplethysmography data acquired during sleep. The method may include detecting the sleep based on the photoplethysmography data. The user data may include demographic data for the user. The user data may include one or more descriptive statistics for heart rate zones of the user during activity derived from the physiological data. The supplemental data may include Global Positioning System (GPS) data acquired for the user during a run.

The second model may estimate the maximal oxygen consumption based in part on a portion of the user data acquired during the run and the GPS data acquired during the run. The second model may include a second regression model. The method may include transitioning to the first model when the supplemental data reaches a predetermined age. Transitioning to the first model may include using the first model with an adjustment based on an historical estimate obtained using the second model. The supplemental data may include a clinical measurement of the maximal oxygen consumption. The second model may adjust a result of the first model according to the clinical measurement of the maximal oxygen consumption. The second model may adjust a result of the first model according to a difference between the clinical measurement of the maximal oxygen consumption and the maximal oxygen consumption calculated according to the first model within one week of the clinical measurement of the maximal oxygen consumption.

In another aspect, there is disclosed herein a computer program product comprising computer-executable code embodied in a non-transitory computer-readable medium that, when executing on one or more computing devices, causes the one or more computing devices to perform the steps of: receiving user data from one or more data sources for a user; providing a plurality of models for estimating a maximal oxygen consumption for the user, each of the models adapted to an availability of data in at least one of the data sources; selecting one of the plurality of models based on the availability of data in the one or more data sources; and estimating the maximal oxygen consumption for the user based on a selected one of the plurality of models.

The user data may include physiological data from a wearable monitor providing physiological data while worn by the user. The user data may include GPS data for the user acquired during a run by the user. The user data may include a clinical measurement of maximal oxygen consumption. The one or more data sources may include a wearable physiological monitor, a GPS device, and a clinical evaluation.

In another aspect, a system disclosed herein includes a physiological monitor configured to continuously acquire heart rate data from a user; a memory storing a plurality of models for estimating maximal oxygen consumption for the user, each of the models adapted to an availability of data in one or more data sources for the user; and a computing device configured by computer-executable code to estimate the maximal oxygen consumption for the user by: receiving user data from at least one of the one or more data sources for the user, selecting one of the plurality of models based on the availability of data in the at least one of the one or more data sources, and estimating the maximal oxygen consumption for the user based on a selected one of the plurality of models and the user data. The computing device may be configured to transmit the maximal oxygen consumption to a user device for display.

### BRIEF DESCRIPTION OF THE DRAWINGS

The foregoing and other objects, features, and advantages of the devices, systems, and methods described herein will be apparent from the following description of particular embodiments thereof, as illustrated in the accompanying drawings. The drawings are not necessarily to scale, emphasis instead being placed upon illustrating the principles of the devices, systems, and methods described herein. In the drawings, like reference numerals generally identify corresponding elements.

FIG. 1 shows a physiological monitoring device.

FIG. 2 shows a physiological monitoring system.

FIG. 3 shows a sensing system.

FIG. 4A shows examples of physiological monitoring devices.

FIG. 4B shows examples of physiological monitoring devices.

FIG. 4C shows examples of physiological monitoring devices.

FIG. 5 shows a smart garment system.

FIG. 6 illustrates a strategy for selecting among modes to calculate VO2 Max.

FIG. 7 illustrates a system for estimating maximal oxygen consumption.

FIG. 8 is a flow chart of a method for estimating maximal oxygen consumption.

### DESCRIPTION

The embodiments will now be described more fully hereinafter with reference to the accompanying figures in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately,” or the like, when accompanying a numerical value, are to be construed as indicating a deviation as would be appreciated by one of ordinary skill in the art to operate satisfactorily for an intended purpose. Similarly, words of approximation such as “approximately” or “substantially” when used in reference to physical characteristics, should be understood to contemplate a range of deviations that would be appreciated by one of ordinary skill in the art to operate satisfactorily for a corresponding use, function, purpose, or the like. Ranges of values and/or numeric values are provided herein as examples only, and do not constitute a limitation on the scope of the described embodiments. Where ranges of values are provided, they are also intended to include each value within the range as if set forth individually, unless expressly stated to the contrary. The use of any and all examples, or exemplary language (“e.g.,” “such as,” or the like) provided herein, is intended merely to better describe the embodiments and does not pose a limitation on the scope of the embodiments. No language in the specification should be construed as indicating any unclaimed element as essential to the practice of the embodiments.

In the following description, it is understood that terms such as “first,” “second,” “top,” “bottom,” “up,” “down,” “above,” “below,” and the like, are words of convenience and are not to be construed as limiting terms unless specifically stated to the contrary.

The term “user” as used herein, refers to any type of animal, human or non-human, whose physiological information may be monitored using an exemplary wearable physiological monitoring device and/or system.

The term “continuous,” as used herein in connection with heart rate data, refers to the acquisition of heart rate data at a sufficient frequency to enable detection of individual heartbeats, and also refers to the collection of heart rate data over extended periods, such as an hour, a day or more (including acquisition throughout the day and night), etc. More generally, with respect to physiological signals that might be monitored by a wearable device, “continuous” or “continuously” will be understood to mean continuously at a rate and duration suitable for the intended time-based processing, and physically at an inter-periodic rate (e.g., multiple times per heartbeat, respiration, and so forth) sufficient for resolving the desired physiological characteristics, such as heart rate, heart rate variability, heart rate peak detection, pulse shape, and so forth. Continuous monitoring should also be understood to include periodic sampling at any suitable interval, duration, and frequency. Thus, for example, continuous monitoring may include measuring a user's body temperature once every ten minutes, or monitoring heart activity by alternately sampling the heart rate for a minute and then pausing sampling for a minute, e.g., to conserve power or memory at times when the measured heart rate indicates that the user is at rest. Sampling may also be dynamic based on sensor input, for example, increasing the sampling rate when signal variability increases, or during periods of relatively higher motion, or based on user input.

At the same time, continuous monitoring is not intended to exclude ordinary data acquisition interruptions, such as temporary displacement of monitoring hardware due to sudden movements, changes in external lighting, loss of electrical power, physical manipulation and/or adjustment by a wearer, physical displacement of monitoring hardware due to external forces, and so forth. It will also be noted that heart rate data or a monitored heart rate, in this context, may more generally refer to raw sensor data, such as optical intensity signals, or processed data therefrom, such as heart rate data, signal peak data, heart rate variability data, or any other physiological or digital signal suitable for recovering heart rate information as contemplated herein. Furthermore, such heart rate data may generally be captured over some historical period that can be subsequently correlated to various other data or metrics related to, e.g., sleep states, recognized exercise activities, resting heart rate, maximum heart rate, and so forth.

The term “computer-readable medium,” as used herein, refers to a non-transitory storage media, such as storage hardware, storage devices, computer memory that may be accessed by a controller, a microcontroller, a microprocessor, a computational system, or the like, or any other module or component of a computational system to encode thereon computer-executable instructions, software programs, and/or other data. The “computer-readable medium” may be accessed by a computational system or a module of a computational system to retrieve and/or execute the computer-executable instructions or software programs encoded on the medium. The non-transitory computer-readable media may include, but are not limited to, one or more types of hardware memory, non-transitory tangible media (for example, one or more magnetic storage disks, one or more optical disks, one or more USB flash drives), virtual or physical computer system memory, physical memory hardware, such as random access memory (such as DRAM, SRAM, EDO RAM), and so forth. Although not depicted, any of the devices or components described herein may include a computer-readable medium or other memory for storing program instructions, data, and the like.

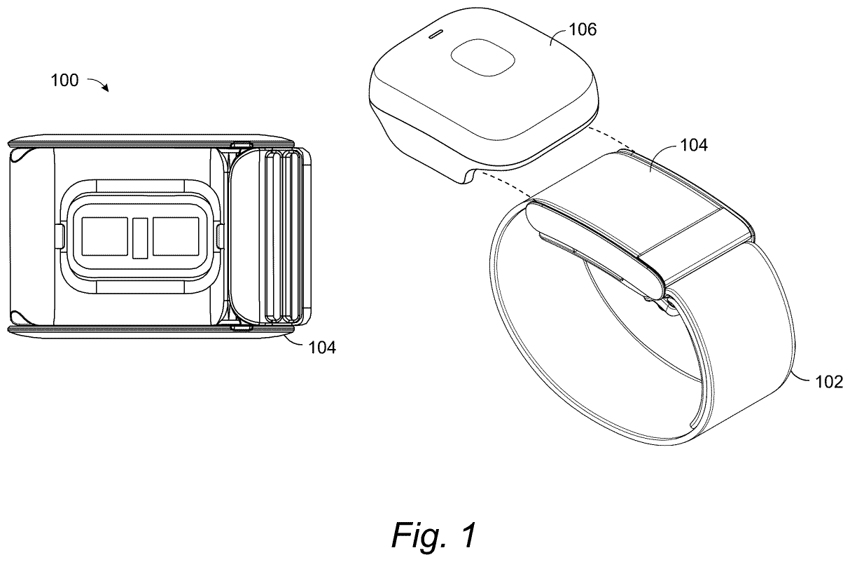

FIG. 1 shows a physiological monitoring system. The system 100 may include a wearable monitor 104 that is configured for physiological monitoring. The system 100 may also include a removable and replaceable battery 106 for recharging the wearable monitor 104. The wearable monitor 104 may include a strap 102 or other retaining system(s) for securing the wearable monitor 104 in a position on a wearer's body for the acquisition of physiological data as described herein. For example, the strap 102 may include a slim elastic band formed of any suitable elastic material such as a rubber or a woven polymer fiber such as a woven polyester, polypropylene, nylon, spandex, and so forth. The strap 102 may be adjustable to accommodate different wrist sizes, and may include any latches, hasps, or the like to secure the wearable monitor 104 in an intended position for monitoring a physiological signal. While a wrist-worn device is depicted, it will be understood that the wearable monitor 104 may be configured for positioning in any suitable location on a user's body, based on the sensing modality and the nature of the signal to be acquired. For example, the wearable monitor 104 may be configured for use on a wrist, a forearm, an ankle, a lower leg, a bicep, a chest, side torso, back, a gluteus, behind the ear, forehead, or any other suitable location(s), and the strap 102 may be, or may include, a waistband or other elastic band or the like within an article of clothing or accessory. In another aspect, the wearable monitor 104 may be configured as a ring, earring, stick-on, clip-on, head-mounted (e.g., glasses or goggles), or other article of clothing or accessory that can be worn by a user, and that contains suitable instrumentation, memory, and/or processing for physiological monitoring as described herein. The wearable monitor 104 may also or instead be structurally configured for placement on or within a garment, e.g., permanently or in a removable and replaceable manner. To that end, the wearable monitor 104 may be shaped and sized for placement within a pocket, slot, and/or other housing that is coupled to or embedded within a garment. In such configurations, the pocket or other retaining arrangement on the garment may include sensing windows or the like so that the wearable monitor 104 can operate while placed for use in the garment. U.S. Pat. No. 11,185,292 and U.S. Pat. Pub. No. 2024/0106283 describe non-limiting example embodiments of suitable wearable monitors 104, and are incorporated herein by reference in their entirety. And while the present disclosure may refer to a wrist-worn wearable or other wearable, it should be understood that any of the other locations or forms described herein are also included unless expressly stated to the contrary or otherwise clear from the context.

The system 100 may include any hardware components, subsystems, and the like to support various functions of the wearable monitor 104 such as data collection, processing, display, and communications with external resources. For example, the system 100 may include hardware for a heart rate monitor using, e.g., photoplethysmography, electrocardiography, or any other technique(s). The system 100 may be configured such that, when the wearable monitor 104 is placed for use about a wrist (or at some other body location), the system 100 initiates acquisition of physiological data from the wearer. In some embodiments, the pulse or heart rate may be acquired optically based on a light source (such as light emitting diodes (LEDs)) and optical detectors in the wearable monitor 104. The LEDs may be positioned to direct illumination toward the user's skin, and optical detectors such as photodiodes may be used to capture illumination intensity measurements indicative of illumination from the LEDs that is reflected and/or transmitted by or through the wearer's skin, or depending on the configuration, through capillaries or arteries.

The system 100 may be configured to record other physiological and/or biomechanical parameters, including, but not limited to, skin temperature (using a thermometer), galvanic skin response (using a galvanic skin response sensor), motion (using one or more multi-axes accelerometers and/or gyroscopes), blood pressure (via physical pressure measurements or other means), sound, electrocardiograms, and the like, as well as environmental or contextual parameters such as ambient light, ambient temperature, humidity, time of day, location, and so forth. For example, the wearable monitor 104 may include sensors such as accelerometers and/or gyroscopes for motion detection, sensors for environmental temperature sensing, sensors to measure electrodermal activity (EDA), sensors to measure galvanic skin response (GSR) sensing, and so forth. The system 100 may also or instead include other systems or subsystems supporting additional functions of the wearable monitor 104. For example, the system 100 may include communications systems to support, e.g., near-field communications, proximity sensing, touch sensing (e.g., via capacitive or resistive sensors), Bluetooth communications, Wi-Fi communications, cellular communications, satellite communications, and so forth. The wearable monitor 104 may also or instead include components such as a Global Positioning System (GPS), a display and/or user interface, a clock and/or timer, and so forth.

The wearable monitor 104 may include one or more sources of battery power, such as a first battery within the wearable monitor 104 and a second battery 106 that is removable from and replaceable to the wearable monitor 104 in order to recharge the battery in the wearable monitor 104. The wearable monitor 104 may also or instead include systems for energy harvesting via, e.g., kinetic energy capture, ambient electromagnetic radiation capture, solar/optical energy capture, and so forth, as well as systems for short and/or medium-range wireless energy transfer to receive power from nearby wireless power sources. Also or instead, the system 100 may include a plurality of wearable monitors 104 (and/or other physiological monitors) that can share battery power or provide power to one another, e.g., using a garment power infrastructure, wireless power sharing network, or the like. The system 100 may perform numerous functions related to continuous monitoring, such as automatically detecting when the user is asleep, awake, exercising, and so forth, and such detections may be performed locally at the wearable monitor 104 or at a remote service such as a mobile device or cloud computing resource coupled in a communicating relationship with the wearable monitor 104 and receiving data therefrom. In general, the system 100 may support continuous, independent monitoring of a physiological signal such as a heart rate, and the underlying acquired data may be stored on the wearable monitor 104 for an extended period until it can be uploaded to a remote processing resource for more computationally complex analysis. In one aspect, the wearable monitor 104 may be a wrist-worn photoplethysmography device, although other form factors are also or instead possible as described herein, such as a ring, a bicep band, a calf band, an elastic band in a garment, a patch, a clip-on device, and so forth.

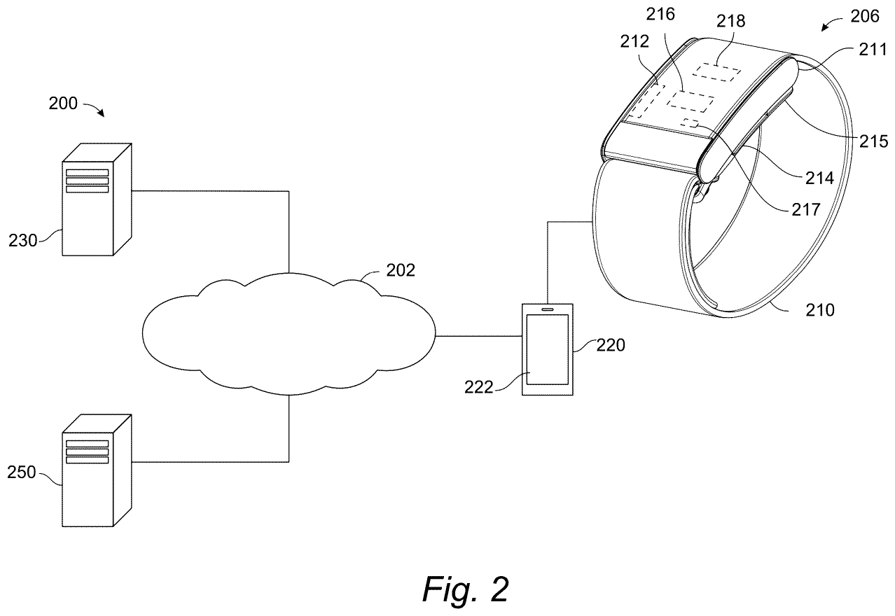

FIG. 2 illustrates a physiological monitoring system. More specifically, FIG. 2 illustrates a physiological monitoring system 200 that may be used with any of the methods or devices described herein. In general, the system 200 may include a physiological monitor 206, a user device 220, a remote server 230 with a remote data processing resource (such as any of the processors or processing resources described herein), and one or more other resources 250, all of which may be interconnected through a data network 202.

The data network 202 may be any of the data networks described herein. For example, the data network 202 may be any network(s) or internetwork(s) suitable for communicating data and information among participants in the system 200. This may include public networks such as the Internet, private networks, telecommunications networks such as the Public Switched Telephone Network or cellular networks using third generation (e.g., 3G or IMT-200), fourth generation (e.g., LTE (E-UTRA) or WiMAX-Advanced (IEEE 802.16m)), fifth generation (e.g., 5G), and/or other technologies, as well as any of a variety of corporate area or local area networks and other switches, routers, hubs, gateways, and the like that might be used to carry data among participants in the system 200. This may also include local or short-range communications infrastructure suitable, e.g., for coupling the physiological monitor 206 to the user device 220, or otherwise supporting communication with local resources. By way of non-limiting examples, short-range communications may include Wi-Fi communications, Bluetooth communications, infrared communications, near field communications, communications with RFID tags or readers, and so forth.

The physiological monitor 206 may, in general, be any physiological monitoring device or system, such as any of the wearable monitors or other monitoring devices or systems described herein. In one aspect, the physiological monitor 206 may be a wearable physiological monitor shaped and sized to be worn on a wrist or other body location. The physiological monitor 206 may include a wearable housing 211, a network interface 212, one or more sensors 214, one or more light sources 215, a processor 216, a haptic device 217 or other user input/output hardware, a memory 218, and a strap 210 for retaining the physiological monitor 206 in a desired location on a user. In one aspect, the physiological monitor 206 may be configured to acquire heart rate data and/or other physiological data from a wearer in an intermittent or substantially continuous manner. In another aspect, the physiological monitor 206 may be configured to support extended, continuous acquisition of physiological data, e.g., for several days, a week, or more.

The network interface 212 of the physiological monitor 206 may be configured to couple the physiological monitor 206 to one or more other components of the system 200 in a communicating relationship, either directly, e.g., through a cellular data connection or the like, or indirectly through a short-range wireless communications channel coupling the physiological monitor 206 locally to a wireless access point, router, computer, laptop, tablet, cellular phone, or other device that can locally process data, and/or relay data from the physiological monitor 206 to the remote server 230 or other resource(s) 250 as necessary or helpful for acquiring and processing data from the physiological monitor 206. The network interface 212 may also or instead facilitate connections among multiple wearable devices, power sources, and the like, e.g., in a wearable device area network or other multi-device monitoring infrastructure.

The one or more sensors 214 may include any of the sensors described herein, or any other sensors or sub-systems suitable for physiological monitoring or supporting functions. By way of example and not limitation, the one or more sensors 214 may include one or more of a light source (including, e.g., LEDs or other wavelength-specific sources of green light, red light, infrared light, and so forth, as well as broadband illumination), an optical sensor, an accelerometer, a gyroscope, a temperature sensor, a galvanic skin response sensor, a capacitive sensor, a resistive sensor, an environmental sensor (e.g., for measuring ambient temperature, humidity, lighting, and the like), a geolocation sensor, and so forth. The one or more sensors 214 may also or instead include sensors (and accompanying hardware/software) for, e.g., a Global Positioning System, a proximity sensor, an RFID tag reader, an RFID tag, a temporal sensor, an electrodermal activity sensor, an electrocardiogram, a pressure sensor, an acoustic sensor (e.g., a microphone), a camera (e.g., visible light and/or infrared), and the like. The one or more sensors 214 may be disposed in the wearable housing 211, or otherwise positioned and configured for physiological monitoring or other functions described herein. In one aspect, the one or more sensors 214 include a light detector configured to provide light intensity data to the processor 216 (or to the remote server 230) for calculating a heart rate and a heart rate variability. The one or more sensors 214 may also or instead include an accelerometer, gyroscope, and the like configured to provide motion data to the processor 216, e.g., for detecting activities such as a sleep state, a resting state, a waking event, exercise, and/or other user activity. In an implementation, the one or more sensors 214 may include a sensor to measure a galvanic skin response of the user. The one or more sensors 214 may also or instead include electrodes or the like for capturing electronic signals, e.g., to obtain an electrocardiogram and/or other electrically-derived physiological measurements.

The processor 216 and memory 218 may be any of the processors and memories described herein. In one aspect, the memory 218 may store physiological data obtained by monitoring a user with the one or more sensors 214, and/or any other sensor data, program data, or other data useful for operation of the physiological monitor 206 or other components of the system 200. It will be understood that, while only the memory 218 on the physiological monitor is illustrated, any other device(s) or components of the system 200 may also or instead include a memory to store program instructions, raw data, processed data, user inputs, and so forth. In one aspect, the processor 216 of the physiological monitor 206 may be configured to obtain heart rate data from the user, such as heart rate data including or based on the raw data from the sensors 214. The processor 216 may also or instead be configured to determine, or assist in a determination of, a condition of the user related to, e.g., health, fitness, strain, recovery, sleep, or any of the other conditions described herein.

The one or more light sources 215 may be coupled to the wearable housing 211 and controlled by the processor 216. At least one of the light sources 215 may be directed toward the skin of a user adjacent to the wearable housing 211. Light from the light source 215, or more generally, light at one or more wavelengths of the light source 215, may be detected by one or more of the sensors 214, and processed by the processor 216 as described herein.

The system 200 may further include a remote data processing resource executing on a remote server 230. The remote data processing resource may include any of the processors and related hardware described herein, and may be configured to receive data transmitted from the memory 218 of the physiological monitor 206, and to process the data to detect or infer physiological signals of interest such as heart rate, heart rate variability, respiratory rate, pulse oxygen, blood pressure, and so forth. The remote server 230 may also or instead evaluate a condition of the user such as a recovery state, sleep state, exercise activity, exercise type, sleep quality, daily activity strain, and any other health or fitness conditions that might be detected based on such data.

The system 200 may include one or more user devices 220, which may work together with the physiological monitor 206, e.g., to provide a display, or more generally, user input/output, for user data and analysis, and/or to provide a communications bridge from the network interface 212 of the physiological monitor 206 to the data network 202 and the remote server 230. For example, the physiological monitor 206 may communicate locally with a user device 220, such as a smartphone of a user, via short-range communications, e.g., Bluetooth, or the like, for the exchange of data between the physiological monitor 206 and the user device 220, and the user device 220 may in turn communicate with the remote server 230 via the data network 202 in order to forward data from the physiological monitor 206 and to receive analysis and results from the remote server 230 for presentation to the user. In one aspect, the user device(s) 220 may support physiological monitoring by processing or pre-processing data from the physiological monitor 206 to support extraction of heart rate or heart rate variability data from raw data obtained by the physiological monitor 206. In another aspect, computationally intensive processing may advantageously be performed at the remote server 230, which may have greater memory capabilities and processing power than the physiological monitor 206 and/or the user device 220.

The user device 220 may include any suitable computing device(s) including, without limitation, a smartphone, a desktop computer, a laptop computer, a network computer, a tablet, a mobile device, a portable digital assistant, a cellular phone, a portable media or entertainment device, or any other computing devices described herein, including, e.g., supplemental wearable devices and/or computers. The user device 220 may provide a user interface 222 for access to data and analysis by a user, and/or to support user control of operation of the physiological monitor 206. The user interface 222 may be maintained by one or more applications executing locally on the user device 220, or the user interface 222 may be remotely served and presented on the user device 220, e.g., from the remote server 230 or the one or more other resources 250.

In general, the remote server 230 may include data storage, a network interface, and/or other processing circuitry. The remote server 230 may process data from the physiological monitor 206 and perform physiological and/or health monitoring/analyses or any of the other analyses described herein, (e.g., analyzing sleep, determining strain, assessing recovery, and so on), and may host a user interface for remote access to this data, e.g., from the user device 220. The remote server 230 may include a web server or other programmatic front end that facilitates web-based access by the user devices 220 or the physiological monitor 206 to the capabilities of the remote server 230 or other components of the system 200.

The system 200 may include other resources 250, such as any resources that can be usefully employed in the devices, systems, and methods as described herein. For example, these other resources 250 may include other data networks, databases, processing resources, cloud data storage, data mining tools, computational tools, data monitoring tools, algorithms, and so forth. In another aspect, the other resources 250 may include one or more administrative or programmatic interfaces for human actors such as programmers, researchers, annotators, editors, analysts, coaches, and so forth, to interact with any of the foregoing. The other resources 250 may also or instead include any other software or hardware resources that may be usefully employed in the networked applications as contemplated herein. For example, the other resources 250 may include payment processing servers or platforms used to authorize payment for access, content, or option/feature purchases. In another aspect, the other resources 250 may include certificate servers or other security resources for third-party verification of identity, encryption or decryption of data, and so forth. In another aspect, the other resources 250 may include a desktop computer or the like co-located (e.g., on the same local area network with, or directly coupled to through a serial or USB cable) with a user device 220, wearable strap 210, or remote server 230. In this case, the other resources 250 may provide supplemental functions for components of the system 200 such as firmware upgrades, user interfaces, and storage and/or pre-processing of data from the physiological monitor 206 before transmission to the remote server 230.

The other resources 250 may also or instead include one or more web servers that provide web-based access to and from any of the other participants in the system 200. While depicted as a separate network entity, it will be readily appreciated that the other resources 250 (e.g., a web server) may also or instead be logically and/or physically associated with one of the other devices described herein, and may, for example, include or provide a user interface 222 for web access to the remote server 230 or a database or other resource(s) to facilitate user interaction through the data network 202, e.g., from the physiological monitor 206 or the user device 220.

In another aspect, the other resources 250 may include fitness equipment or other fitness infrastructure. For example, a strength training machine may automatically record repetitions and/or added weight during repetitions, which may be wirelessly accessible by the physiological monitor 206 or some other user device 220. More generally, a gym may be configured to track user movement from machine to machine, and report activity from each machine in order to track various strength training activities in a workout. The other resources 250 may also or instead include other monitoring equipment or infrastructure. For example, the system 200 may include one or more cameras to track motion of free weights and/or the body position of the user during repetitions of a strength training activity or the like, and/or the cameras may be integrated into the physiological monitor 206 or other user device 220. Similarly, a user may wear, or have embedded in clothing, tracking fiducials such as visually distinguishable objects for image-based tracking, or radio beacons or the like for other tracking. In another aspect, weights may themselves be instrumented, e.g., with sensors to record and communicate detected motion, and/or beacons or the like to self-identify type, weight, and so forth, in order to facilitate automated detection and tracking of exercise activity with other connected devices.

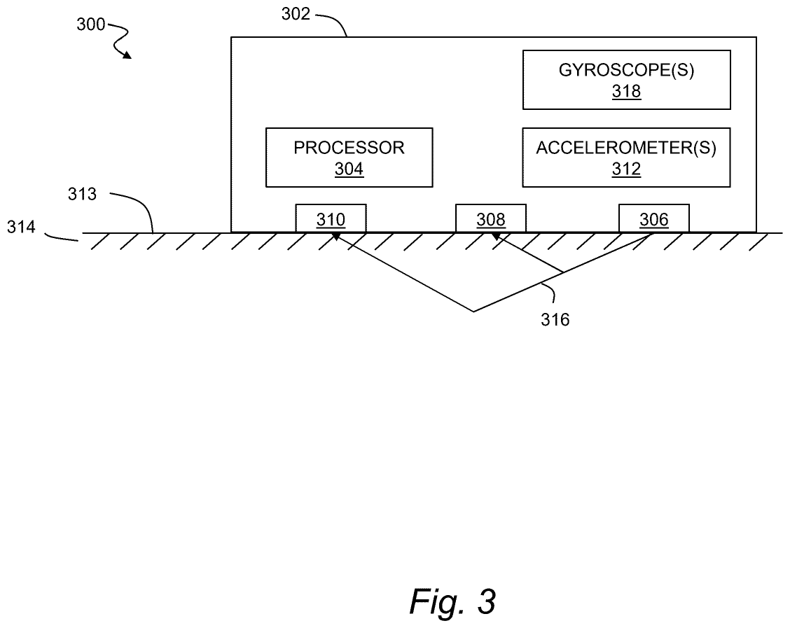

FIG. 3 shows a sensing system. In general, the system 300 may include a physiological monitor 302 with a processor 304, a light source 306, a first sensor 308 (e.g., a first photodetector), a second sensor 310 (e.g., a second photodetector), one or more accelerometers 312, one or more gyroscopes 318, and any other hardware or other components and systems suitable for physiological monitoring as described herein. The physiological monitor 302 may be positioned for use against a surface 313 of the skin 314 of a user where the light source 306 and sensors 308, 310 can contact the skin 314 for acquisition of physiological data. Although not depicted, it will be understood that the physiological monitor 302 may generally be retained in position using any of the straps, garments, patches, bands, clamps, clips, or the like described herein, and/or integrated into other wearable garments, accessories, and the like, such as audio earbuds, earrings or similar, glasses and/or other eyewear, a ring, a headband, and so forth.

The processor 304 may be any microprocessor, microcontroller, application-specific integrated circuit, or other processing circuitry or combination of the foregoing suitable for controlling operation of the physiological monitor and acquiring physiological data.

The light source 306 may include one or more light-emitting diodes or other sources of illumination, and may be positioned within the physiological monitor 302 such that, when the physiological monitor 302 is placed for use on the skin 314, the light source 306 directs illumination toward the skin 314 and the illumination is reflected back toward the sensors 308, 310 as indicated by arrows 316 (or transmitted through the tissue to one or more opposing sensors), where the intensity can be measured. In one aspect, the light source 306 may include light-emitting diodes that emit light in the green, red, infrared, near-infrared, or other suitable wavelength ranges, which can provide desired light transmission through human skin, facilitating low-power transmission of measurable illumination to the sensors 308, 310, although other illumination sources and wavelengths may also or instead be used.

The sensors 308, 310 may be oriented to contact the skin 314 when the physiological monitor 302 is placed for use on this skin 314, and positioned so that the sensors 308, 310 can capture illumination reflected and/or transmitted by the skin from the light source 306. In general, the sensors 308, 310 may include photodiodes, photodetectors, or any other sensor(s) responsive to illumination from the light source 306. This may include broadband optical sensors, narrowband optical sensors, filtered sensors, or the like. In general, a first sensor 308 may be positioned closer to the light source 306 than a second sensor 310 to facilitate detection of differential intensity in the measured wavelength(s). For example, the first sensor 308 may be positioned 1-4 millimeters from the light source 306 and the second sensor 310 may be positioned 2-8 millimeters from the light source, or about twice as far as the first sensor 308 from the light source 306.

Other spacings may also or instead be used depending on, e.g., the intensity of the light source 306, the sensitivity of the sensors 308, 310, the contact force of the physiological monitor 302 on the skin 314, the degree of incursion of ambient light, the physiological measurements/properties of interest, and so forth. In one aspect, the sensors 308, 310 may be linearly arranged in a straight line away from the light source 306. While this provides consistency in comparative measurements, it is not strictly required, and the sensors 308, 310 may be displaced in any of a number of directions away from the light source 306 provided they both contact the skin 314 in a manner that permits capture of light through the skin 314 from the light source 306. In another aspect, the physiological monitor 302 may include one or more other light sources and/or light sensors, which may be arranged to improve accuracy and/or provide redundancy for the contact detection, or to support other measurements such as oxygenation or skin thickness. This may include light sources/sensors using different ranges of wavelengths, different patterns of illumination, and so forth. In another aspect, the two sensors 308, 310 may be positioned at different distances from a perimeter of the physiological monitor 302 so that the sensors 308, 310 can acquire differential intensity values for ambient light incident on the skin and transmitted through the skin to the sensors 308, 310.

In operation, the processor 304 may acquire raw intensity data from the sensors 308, 310, and perform local calculations such as pre-processing raw data for heart rate measurements, or evaluating whether the physiological monitor 302 is properly placed for use on the skin 314.

The accelerometer 312 may include, e.g., one or more single axis or multi-axis accelerometers, which may usefully measure motion of the physiological monitor 302 to support calculations such as automated activity detection, device on/off evaluation, degree of musculoskeletal activation, and so forth. Other motion and orientation sensing hardware—such as one or more gyroscopes 318, inertial motion sensors, and/or other micro-electromechanical system (MEMS) sensors—may also or instead be used for these purposes. More generally, the physiological monitor 302 may include any additional components, subsystems, and the like suitable for supporting various modes of physiological monitoring and contextual data acquisition as described herein.

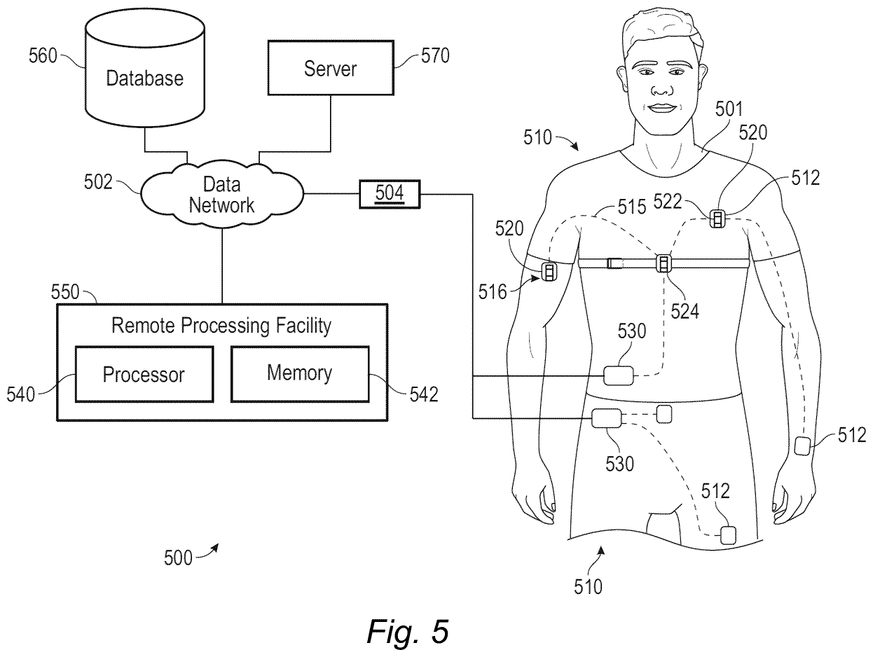

The physiological monitors described herein—e.g., in the systems 100, 200, 300 described above or elsewhere herein—may be provided in one or more different form factors. That is, although a wrist-worn device is illustrated in FIGS. 1 and 2, and garments with sensors are illustrated in FIG. 5, other form factors are also or instead possible, some of which are discussed below by way of example.

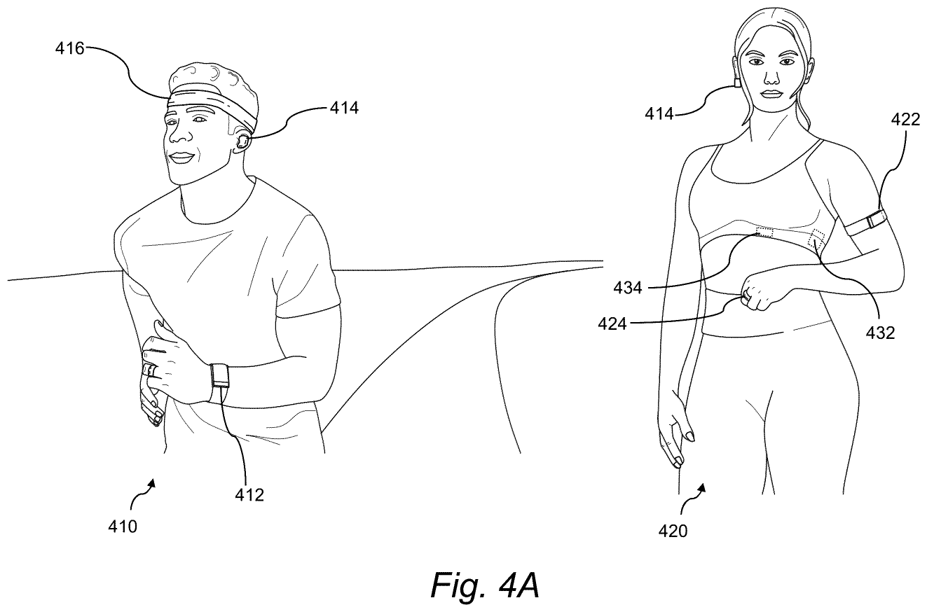

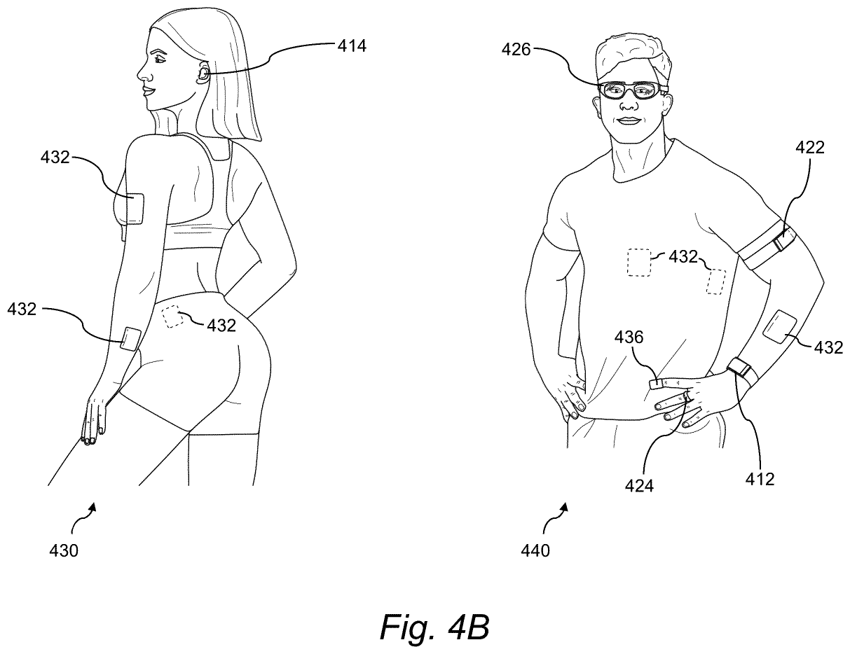

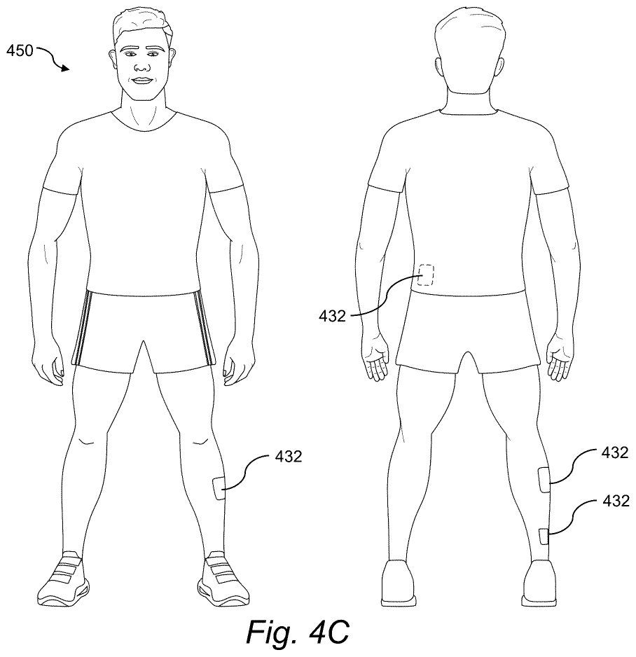

FIGS. 4A-4C illustrate physiological monitoring devices. The illustrated devices may include any of the hardware, software, and/or other components described herein for physiological sensing and/or other functions, and may be embodied in various form factors for various use cases. These various form factors may be used individually or as multiple independent or cooperating physiological monitoring devices, and may include two or more devices of the same type (e.g., two wrist-worn devices, two or more patches, and so on), and/or two or more different types of devices. Moreover, other form factors, and combinations thereof, may also or instead be used for physiological monitoring as described herein. It will further be understood that each of the different example form factors shown in these figures or elsewhere herein may include any one or more of the various sensors, emitters, processors, memories, interfaces, power supplies, and/or other processing and control circuitry, including without limitation any of the foregoing described herein, e.g., with reference to FIGS. 1-3 above.

FIG. 4A shows a first user 410 and a second user 420. The first user 410 may be wearing one or more physiological monitors, such as a wrist-worn device 412 (such as any described herein), an ear-worn device 414 (including on-ear devices retained with a clamp, clip, or other mechanism, and/or in-ear devices, such as earbuds or the like, that are retained at least in part within the ear canal), and a headband 416 or similar.

In one aspect, an ear-worn device 414 may be structurally configured to be partially or entirely inserted within an ear canal of the first user 410. In another aspect, the ear-worn device 414 may be configured to be worn on the ear lobe, or in some other location on the ear where, e.g., temperature, blood flow, respiration, and/or other physiological parameters can be measured. In one aspect, an ear-worn device 414 may be configured for heart rate monitoring, such as any of the heart rate monitoring described herein. For example, this may include continuous heart rate monitoring with optical sensors based on changes in blood volume beneath the skin. The ear-worn device 414 may also or instead be configured for temperature monitoring. For example, the ear-worn device 414 may include one or more infrared sensors, thermistors, thermocouples, or the like to measure the temperature of the ear canal and/or other surfaces. Surface measurements may also or instead be used to support other inferences about body temperature, heat dissipation, and the like, which may be related to current activity levels, general health and wellness, and so forth.

In another aspect, the ear-worn device 414, or any of the other devices described herein, may be configured for activity tracking. For example, the ear-worn device 414 may include one or more accelerometers, gyroscopes, Global Positioning System (GPS) sensors, and so forth to detect motion and provide information about physical activity levels. This may, for example, include large-scale motion, such as geographical movement and elevation changes, that can be tracked with GPS or the like, or local movement detected by the ear-worn device 414, which may be tracked with multi-axis gyroscopes, multi-axis accelerometers, and so forth. These latter sensors may be used to infer, e.g., steps taken, gait analysis, activity type, activity level, and/or overall movement.

The ear-worn device 414, or any of the other devices described herein, may also or instead be configured for blood pressure monitoring. This may, for example, include techniques based on cardiovascular waveform analysis (e.g., using the shape of a PPG or ECG signal from a single location), pulse transit time (e.g., based on the time difference between waveforms at two or more physical locations on the body with two or more monitors), pulse wave velocity (similar to pulse transit time, but over longer arterial distances), physical pulse monitoring (e.g., with pressure sensors, haptic stimulus responses, or other mechanical and/or dynamic techniques), tonometry (measuring the force required to counteract arterial pressure), oscillometric measurement (measuring oscillations in the arterial wall as a cuff deflates around a region of interest), volume clamping (measuring changes in pressure that are required to maintain constant blood volume in a region of interest), and so forth. Some of these blood pressure monitoring techniques are better suited to specific types and locations of monitors and may be more suited to, e.g., wrist bands, bicep bands, chest straps, finger rings, and so forth, but are included here for completeness.

The ear-worn device 414, or any of the other devices described herein, may also or instead be configured for electrodermal activity (EDA) monitoring. For example, the ear-worn device 414 may include one or more electrodes in contact with the skin, which may be used to measure the electrical conductance thereof, and to infer, e.g., sweat levels, skin hydration, and/or other parameters correlated to skin conductance. Electrodes may also or instead be used for, e.g., ECG monitoring or the like.

The ear-worn device 414, or any of the other devices described herein, may also or instead be configured to sense blood oxygen saturation (also referred to as pulse oximetry or SpO2) monitoring. To this end, the ear-worn device 414 may include one or more optical sources and detectors, and the system may use different absorption spectra of oxygenated and deoxygenated hemoglobin to estimate pulse oxygen saturation. In another aspect, the ear-worn device 414, or any of the other devices described herein, may be configured for brainwave monitoring, e.g., using electroencephalogram (EEG) sensors to monitor brainwave activity.

The ear-worn device 414, or any of the other devices described herein, may also or instead be configured for respiration rate monitoring. In one aspect, respiration rate may be inferred using respiratory sinus arrhythmia or other techniques to infer respiration rate from a measured heart rate signal over time. In another aspect, respiration rate may be inferred from physical changes in the ear canal (or chest, or other body part, where applicable to a particular sensor). Other techniques may also or instead be used. For example, the ear-worn device 414 may include a microphone or other audio transducer, and the respiration rate may be inferred from audio data acquired from the user.

In another aspect, a headband 416 may be structurally and programmatically configured for physiological sensing and/or monitoring using any of the systems and methods described herein. For example, the headband 416 may be configured to monitor heart rate, temperature, brain activity, electromyography, galvanic skin response, motion, activity, and so forth. In general, the sensors and processing may be adapted for the form factor of the headband 416. For example, the headband 416 may use temperature sensors to measure skin temperature and/or ambient temperature around the head. For brain activity, the headband 416 may include EEG sensors or the like embedded within the headband 416 to measure electrical activity in the brain, which can be used for monitoring brain waves associated with different states such as relaxation, concentration, and/or sleep. More generally, any physiological monitoring techniques described herein that can be adapted for use in a corresponding form factor may be deployed, either alone or in combination, for physiological monitoring with the headband 416. In another aspect, the headband 416 may incorporate a brain-computer interface (BCI) for control of a physiological monitoring system. This may, for example, include any system suitable for direct communication between the brain and external devices based on, e.g., signal acquisition using techniques such as electroencephalography, processing of these raw signals, feature extraction and translation, and then command execution based on an inferred user intention.

The second user 420 may be wearing one or more physiological monitors such as an ear-worn device 414 (which may be any as described herein, and which may be configured as a clamp, clip, earring, or similar, as shown), a bicep band 422, a ring 424, a patch 432 (such as any as described herein, e.g., with reference to FIG. 4B), and a band sensor 434.

The bicep band 422 may be configured for physiological monitoring and sensing using any of the systems and methods described herein, e.g., by retaining a sensor in place with the bicep band 422 or integrating components of the sensor into the bicep band 422, or some combination of these. The bicep band 422 may be configured to monitor heart rate, motion, activity, temperature, blood pressure, blood oxygen saturation, hydration, body composition, ultraviolet light exposure, electrodermal activity, and so forth, as well as combinations of the foregoing. In one aspect, electromyography (EMG) may be used to measure electrical activity in the muscles, e.g., with one or more electrical contacts or the like embedded in the bicep band 422, which can provide information about muscle contraction and fatigue during physical activity. Body composition analysis may be performed using, e.g., bioelectrical impedance analysis to estimate various components of body composition such as fat (percentage or mass), muscle (percentage or mass), and hydration. In another aspect, the bicep band 422 may include one or more sensors to measure ambient light, and more specifically, ambient ultraviolet (UV) light. This may be used to monitor UV exposure, and to provide recommendations to the user to meet certain healthy thresholds for, e.g., vitamin D synthesis, mood, and immune function, and/or to provide alerts concerning possible overexposure. In another aspect, the bicep band 422 or other form factors described herein may be adapted for gesture control based on the capture of motion signals and corresponding inferences of user intent. While a bicep band 422 is illustrated, it will be understood that similar bands for other body parts may also or instead be used, such as leg bands (or more specifically, thigh bands, calf bands, ankle bands, etc.), chest bands, abdomen bands, neck bands, wrist bands, and so forth.

The ring 424 may be configured for physiological monitoring and sensing using any of the systems and methods described herein. For example, the ring 424 may be configured to monitor heart rate, motion, activity, sleep, temperature, blood pressure, respiration rate, blood oxygen saturation, hydration, UV exposure, and so forth. A ring 424 is also advantageously positioned to capture a wide range of hand motions, and may be configured for gesture control of physiological monitoring and/or related hardware and software. The ring 424 may be configured for wearing on a finger, as shown in the figure, or another portion of a wearer's body (e.g., a thumb, a toe, and so forth).

The band sensor 434 may be the same or similar to the other monitors described herein and/or any of the bands as described herein. In an aspect, the band sensor 434 may include a monitor inserted into (e.g., placed into a pocket or the like), coupled with, embedded within, or the like, a strap or band, e.g., an elastic band in an article of clothing, an accessory, or similar.

FIG. 4B shows a third user 430 and a fourth user 440. The third user 430 may be wearing one or more physiological monitors such as an ear-worn device 414, which may be the same as or similar to any of those described herein, and one or more patches 432 that include sensors and the like to support physiological monitoring. By way of example, a patch 432 may be configured for physiological monitoring and sensing of heart rate monitoring, temperature, activity, motion, blood pressure, blood oxygen saturation, respiration rate, blood glucose, perspiration, hydration, ultraviolet exposure, and so forth, as well as combinations of the foregoing. In one aspect, the patch 432 may include a continuous glucose monitor with a sensor for insertion into fatty tissue under the skin, along with a transmitter to wirelessly transmit glucose data to a smartphone or other device. In another aspect, the patch 432 may include a hydration monitor using, e.g., electrical impedance analysis to measure resistance and reactance of body tissue with a small electrical current, or bioimpedance spectroscopy to measure impedance at various frequencies of electrical current. Hydration monitoring may also or instead use a wearable patch to collect sweat and analyze electrolyte concentrations correlated to hydration. Other techniques for measuring hydration using, e.g., near-infrared spectroscopy or capacitance hygrometry, may also or instead be employed where suitable adaptations can be made to any of the wearable monitors described herein. In another aspect, the patch 432, or any of the other monitors described herein, may be adapted to monitor environmental conditions such as temperature, humidity, air quality, noise, light, and the like that might be used to supplement physiological monitoring when evaluating the condition of a user. In another aspect, the patch 432, or any of the other monitors described herein, may be adapted for electrodermal activity monitoring, e.g., for tracking autonomic nervous system activity, stress, and the like based on galvanic skin response. One or more patches 432 may be coupled to a user in one or more of a plurality of locations on the body, such as those shown on the third user 430—e.g., a portion of an arm (e.g., the upper arm and/or the lower arm), and on or near the gluteus maximus, and similar. Other locations are also or instead possible, such as the chest, the abdomen, the forehead or temples, the wrist, a hand, a finger, a foot, a neck, a backside, the pelvic region, a portion of the back, a portion of a leg, and so forth.

The fourth user 440 may be wearing one or more physiological monitors such as a bicep band 422, a wrist-worn device 412, a ring 424, and a patch 432, which may be the same or similar to any of the monitors described herein. The fourth user 440 further is shown with eyewear 426 and a fingertip monitor 436, as further explained below by way of example.

The eyewear 426 may include sensors or the like in contact areas or similar, such as a temple region, face region (e.g., via the frame or lens), or other head portion of the fourth user 440. For example, the eyewear 426 may be configured for physiological monitoring and sensing of heart rate, temperature, brain activity, motion, activity type, blood pressure, blood oxygen saturation, and so forth, as well as combinations of the foregoing. In one aspect, the eyewear 426 may employ electrooculography (EOG) to measure electrical activity of the muscles around the eyes or another region of the head/face, which can be used, e.g., to track eye movements and provide insights into cognitive states, attention levels, fatigue, and so forth. In another aspect, one or more EEG sensors may be integrated into the frame and/or temples of the eyewear 426 to measure electrical activity in the brain. The eyewear 426 may also or instead be configured to perform eye tracking using cameras and/or infrared or other sensors to monitor movement of the eyes, which can be used for various applications, including human-computer interaction, attention monitoring, and so forth. The eyewear 426 may also or instead be configured for augmented reality (AR) and virtual reality (VR) biometrics, e.g., where the eyewear 426 can include sensors that monitor physiological parameters to enhance user experience and safety, and to visually present information to the user related to any of the foregoing. In another aspect, the eyewear 426 may include cameras, microphones, and the like for recording and tracking environment information.

The fingertip monitor 436 may include a clamp, clip, or the like, and may be the same or similar to any of the physiological monitors described herein. In some respects, the fingertip monitor 436 may include a pulse oximeter configured to measure oxygen saturation and/or heart rate for monitoring respiratory and/or cardiovascular health.

FIG. 4C shows the front and back of a fifth user 450 showing further example locations for a patch 432 or the like as described herein.

More generally, any one or more of the sensing modalities described herein may, provided suitable adaptations can be made, be deployed in any one or more of the wearable devices described herein. Furthermore, one or more of the wearable devices may communicate with one or more other wearable devices and/or with a control device such as a smartphone or other computing device, to perform cooperative monitoring. For example, various monitoring techniques, such as electrocardiography or blood pressure measurements using pulse transit time, may usefully be performed by combining signals from sensors at two or more different body locations, and a control device may usefully acquire signals from multiple devices and locations to perform such analysis. Similarly, multiple motion signals from different body locations may be used to refine activity detection, measure body temperature, and so forth. Thus, in one aspect, two or more wearable devices may cooperate with one another to perform an integrated sensing operation such as any of those described herein.

In another aspect, any one or more of the wearable electronic devices described herein may use energy harvesting to generate power from various external sources, and/or to supplement power supplied by an internal battery or the like. For example, a device may use solar energy harvesting to extract solar energy from ambient light sources. This may include integrating solar cells or other ambient light collectors into the wearable device to capture energy from sunlight and/or artificial light sources. In another aspect, the device may use kinetic energy harvesting to generate energy from movements by a user of the device. In another aspect, the device may use thermal energy harvesting to generate power based on differences between the body of the wearer and the surrounding environment. The device may also or instead use vibration energy harvesting, radio frequency energy harvesting (e.g., by capturing ambient RF signals, such as Wi-Fi or cellular signals, and converting them into usable electrical power), ambient light harvesting, and so forth. Other techniques may also or instead be used to provide external power, such as beam steering or resonant techniques for short-range or medium-range radio frequency power transfers. More generally, any technique or combination of techniques for powering a device, and/or for supplementing an internal power source such as a battery, with power from ambient sources may be used to power one of the monitoring devices described herein.

FIG. 5 shows a smart garment system. One limitation on wearable sensors can be body placement. Devices are typically wrist-based, and may occupy a location that a user would prefer to reserve for other devices or jewelry, or that a user would prefer to leave unadorned for aesthetic or functional reasons. This location also places constraints on what measurements can be taken, and may also limit user activities. For example, a user may be prevented from wearing boxing gloves while wearing a sensing device on their wrist. To address this issue, physiological monitors may also or instead be embedded in clothing, which may be specifically adapted for physiological monitoring with the addition of communications interfaces, power supplies, device location sensors, environmental sensors, geolocation hardware, payment processing systems, and any other components to provide infrastructure and augmentation for wearable physiological monitors. Such “smart garments” offer additional space on a user's body for supporting monitoring hardware, and may further enable sensing techniques that cannot be achieved with single sensing devices. For example, embedding a plurality of physiological sensors or other electronic/communication devices in a shirt may allow electrical sensors to be placed around a torso to support electrocardiogram (ECG) based heart rate measurements, or placed around muscles such as the pectoralis major, latissimus dorsi, biceps brachii, and other major muscle groups to support muscle oxygen saturation measurements. In another aspect, optical sensors may be positioned along an arterial pathway or the like to support pulse transit time measurements for calculation of blood pressure. The infrastructure provided by a garment may also support other supplemental functions beyond physiological monitoring. For example, wireless antennas may be placed above the upper portion of the thoracic spine to achieve desired communications signals, or a contactless payment system may be embedded in a sleeve cuff for interactions with a payment terminal. Smart garments may also free up body surfaces for other devices. For example, if sensors in a wrist-worn device that provide heart rate monitoring and step counting can be instead embedded in a user's undergarments, the user may still receive the biometric information they desire, while also being able to wear jewelry or other accessories for suitable occasions.

The present disclosure generally includes smart garment systems and techniques. It will be understood that a “smart garment” as described herein generally includes a garment that incorporates infrastructure and devices to support, augment, or complement various physiological monitoring modes. Such a garment may include a wired, local communication bus for intra-garment hardware communications, a wireless communication system for intra-garment hardware communications, a wireless communication system for extra-garment communications, and so forth. The garment may also or instead include a power supply, a power management system, processing hardware, data storage, and so forth, any of which may support enriched functions for the smart garment.

In general, the smart garment system 500 illustrated in FIG. 5 may include a plurality of components—e.g., a garment 510, one or more modules 520, a controller 530, a processor 540, a memory 542, and so on—capable of communicating with one another over a data network 502. The garment 510 may be wearable by a user 501 and configured to communicate with a module 520 having a physiological sensor 522 that is structurally configured to sense a physiological parameter of the user 501. As discussed herein, the module 520 may be controllable by the controller 530 based at least in part on a location 516 where the module 520 is located on or within the garment 510. This position-based information may be derived from an interaction and/or communication between the module 520 and the garment 510 using various techniques. It will be understood that, while two controllers 530 are shown, the garment 510 may include a single inter-garment controller, or any number of separate controllers 530 in any number of garments 510 (e.g., one per garment, or one for all garments worn by a person, etc.), and/or controllers may be integrated into other modules 520.

For communication over the data network 502, the system 500 may include a network interface 504, which may be integrated into the garment 510, included in the controller 530, or in some other module or component of the system 500, or some combination of these. The network interface 504 may generally include any combination of hardware and software configured to wirelessly communicate data to remote resources. For example, the network interface 504 may use a local connection to a laptop, smartphone, or the like that couples, in turn, to a wide area network for accessing, e.g., web-based or other network-accessible resources. The network interface 504 may also or instead be configured to couple to a local access point such as a router or wireless access point for connecting to the data network 502. In another aspect, the network interface 504 may be a cellular communications data connection for direct, wireless connection to a cellular network or the like.

The data network 502 may be any as described herein. By way of example, some embodiments of the system 500 may be configured to stream information wirelessly to a social network, a data center, a cloud service, and so forth. In some embodiments, data streamed from the system 500 to the data network 502 may be accessed by the user 501 (or other users) via a website. The network interface 504 may thus be configured such that data collected by the system 500 is streamed wirelessly to a remote processing facility 550, database 560, and/or server 570 for processing and access by the user. In some embodiments, data may be transmitted automatically, without user interaction, for example by storing data locally and transmitting the data over available local area network resources when a local access point such as a wireless access point or a relay device (such as a laptop, tablet, or smartphone) is available. In some embodiments, the system 500 may include a cellular system or other hardware for independently accessing network resources from the garment 510 without requiring local network connectivity. It will be understood that the network interface 504 may include a computing device such as a mobile phone or the like. The network interface 504 may also or instead include or be included on another component of the system 500, or some combination of these. Where battery power or communications resources can advantageously be conserved, the system 500 may preferentially use local networking resources when available, and reserve cellular communications for situations where a data storage capacity of the garment 510 is reaching capacity. Thus, for example, the garment 510 may store data locally up to some predetermined threshold for local data storage, below which data is transmitted over local networks when available. The garment 510 may also transmit data to a central resource using a cellular data network only when local storage of data exceeds the predetermined threshold.

The garment 510 may include one or more designated areas 512 for positioning a module to sense a physiological parameter of the user 501 wearing the garment 510. One or more of the designated areas 512 may be specifically tailored for receiving a module 520 therein or thereon. For example, a designated area 512 may include a pocket structurally configured to receive a module 520 therein. Also or instead, a designated area 512 may include a first fastener configured to cooperate with a second fastener disposed on a module 520. One or more of the first fastener and the second fastener may include at least one of a hook-and-loop fastener, a button, a clamp, a clip, a snap, a projection, and a void.

By placing a pocket or the like in one of these designated areas 512, a position of a module 520 can be controlled, and where an RFID tag, sensor, or the like is used, the designated area 512 can specifically sense when a module 520 is positioned there for monitoring, and can communicate the detected location to any suitable control circuitry.

The garment 510 may also or instead incorporate other infrastructure 515 to cooperate with a module 520. For example, the garment infrastructure 515 may include infrastructure 515 related to ECG devices, such as ECG pads (or otherwise electrically conductive sensor pads and/or electrodes that connect to the module 520, controller 530, and/or another component of the system 500), lead wires, and the like. By way of further example, the garment infrastructure 515 may include wires or the like embedded in the garment 510 to facilitate wired data or power transfer between installed modules 520 and other system components (including other modules 520). The infrastructure 515 may also or instead include integrated features for, e.g., powering modules, supporting data communications among modules, and otherwise supporting operation of the system 500. The infrastructure 515 may also or instead include location or identification tags or hardware, a power supply for powering modules 520 or other hardware, communications infrastructure as described herein, a wired intra-garment network, or supplemental components such as a processor, a Global Positioning System (GPS), a timing device, e.g., for synchronizing signals from multiple garments, a beacon for synchronizing signals among multiple modules 520, and so forth. More generally, any hardware, software, or combination of these suitable for augmenting operation of the garment 510 and a physiological monitoring system using the garment 510 may be incorporated as infrastructure 515 into the garment 510 as contemplated herein.

The modules 520 may generally be sized and shaped for placement on or within the one or more designated areas 512 of the garment 510. For example, in certain implementations, one or more of the modules 520 may be permanently affixed on or within the garment 510. In such instances, the modules 520 may be washable. Also or instead, in certain implementations, one or more of the modules 520 may be removable and replaceable relative to the garment 510. In such instances, the modules 520 need not be washable, although a module 520 may be designed to be washable and/or otherwise durable enough to withstand a prolonged period of engagement with a designated area 512 of the garment 510. A module 520 may be capable of being positioned in more than one of the designated areas 512 of the garment 510. That is, one or more of the plurality of modules 520 may be configured to sense data using a physiological sensor 522 in a plurality of designated areas 512 of the garment 510.

A module 520 may include one or more physiological sensors 522 and a communications interface 524 programmed to transmit data from at least one of the physiological sensors 522. For example, the physiological sensors 522 may include one or more of a heart rate monitor (e.g., one or more PPG sensors or the like), an oxygen monitor (e.g., a pulse oximeter), a blood pressure monitor, a thermometer, an accelerometer, a gyroscope, a position sensor, a Global Positioning System, a clock, a galvanic skin response (GSR) sensor, or any other electrical, acoustic, optical, camera, or other sensor or combination of sensors and the like useful for physiological monitoring, environmental monitoring, or other monitoring as described herein. In one aspect, the physiological sensors 522 may include a conductivity sensor or the like used for electromyography, electrocardiography, electroencephalography, or other physiological sensing based on electrical signals. The data received from the physiological sensors 522 may include at least one of heart rate data and/or similar data related to blood flow (e.g., from PPG sensors), muscle oxygen saturation data, temperature data, movement data, position/location data, environmental data, temporal data, blood pressure data, and so on.

Thus, certain embodiments include one or more physiological sensors 522 configured to provide continuous measurements of heart rate using photoplethysmography or the like. The physiological sensor 522 may include one or more light emitters for emitting light at one or more desired frequencies toward the user's skin, and one or more light detectors for receiving light reflected from the user's skin. The light detectors may include a photo-resistor, a phototransistor, a photodiode, and the like. A processor may process optical data from the light detector(s) to calculate a heart rate based on the measured, reflected light. The optical data may be combined with data from one or more motion sensors, e.g., accelerometers and/or gyroscopes, to minimize or eliminate noise in the heart rate signal caused by motion or other artifacts. The physiological sensor 522 may also or instead provide at least one of continuous motion detection, environmental temperature sensing, electrodermal activity (EDA) sensing, galvanic skin response (GSR) sensing, and the like.

The system 500 may include different types of modules 520. For example, a number of different modules 520 may each provide a particular function. Thus, the garment 510 may house one or more of a temperature module, a heart rate/PPG module, a muscle oxygen saturation module, a haptic module, a wireless communication module, or combinations thereof, any of which may be integrated into a single module 520 or deployed in separate modules 520 that can communicate with one another. Some measurements, such as temperature, motion, optical heart rate detection, and the like, may have preferred or fixed locations, and pockets or fixtures within the garment 510 may be adapted to receive specific types of modules 520 at specific locations within the garment 510. For example, motion may preferentially be detected at or near extremities, while heart rate data may preferentially be gathered near major arteries. In another aspect, some measurements, such as temperature, may be measured anywhere, but may preferably be measured at a single location in order to avoid certain calibration issues that might otherwise arise through arbitrary placement.

In another aspect, the system 500 may include two or more modules 520 placed at different locations and configured to perform differential signal analysis. For example, the rate of pulse travel and the degree of attenuation in a cardiac signal may be detected using two or more modules at two or more locations, e.g., at the bicep and wrist of a user, or at other locations similarly positioned along an artery. These multiple measurements support a differential analysis that permits useful inferences about heart strength, pliability of circulatory pathways, blood pressure, and other aspects of the cardiovascular system that may indicate cardiac age, cardiac health, cardiac conditions, and so forth. Similarly, muscle activity detection might be measured at different locations to facilitate a differential analysis for identifying activity types, determining muscular fitness, and so forth. More generally, multiple sensors can facilitate differential analysis. To facilitate this type of analysis with greater precision, the garment infrastructure may include a beacon or clock for synchronizing signals among multiple modules, particularly where data is temporarily stored locally at each module, or where the data is transmitted to a processor from different locations wirelessly, where packet loss, latency, and the like may present challenges to real-time processing.

The communications interface 524 may be any as described herein, for example, including any of the features of the network interface 504 described above.

The controller 530 may be configured, e.g., by computer-executable code or the like, to determine a location of the module 520. This may be based on contextual measurements, such as accelerometer data from the module 520, which may be analyzed by a machine learning model or the like to infer a body position. In another aspect, this may be based on other signals from the module 520. For example, signals from sensors, such as photodiodes, temperature sensors, resistors, capacitors, and the like, may be used alone or in combination to infer a body position. In another aspect, the location may be determined based on a proximity of a module 520 to a proximity sensor, RFID tag, or the like at or near one of the designated areas 512 of the garment 510. Based on the location, the controller 530 may adapt operation of the module 520 for location-specific operation. This may include selecting filters, processing models, physiological signal detections, and the like. It will be understood that operations of the controller 530, which may be any controller, microcontroller, microprocessor, or other processing circuitry, or the like, may be performed in cooperation with another component of the system 500, such as the processor 540 described herein, one or more of the modules 520, or another computing device. It will also be understood that the controller 530 may be located on a local component of the system 500 (e.g., on the garment 510, in a module 520, and so on) or as part of a remote processing facility 550, or some combination of these. Thus, in an aspect, a controller 530 is included in at least one of the plurality of modules 520. And, in another aspect, the controller 530 is a separate component of the garment 510, and serves to integrate functions of the various modules 520 connected thereto. The controller 530 may also or instead be remote relative to each of the plurality of modules 520, or some combination of these.

The controller 530 may be configured to control one or more of (i) sensing performed by a physiological sensor 522 of the module 520 and (ii) processing by the module 520 of the data received from a physiological sensor 522. That is, in certain aspects, the combination of sensors in the module 520 may vary based on where it is intended to be located on a garment 510. In another aspect, processing of data from a module 520 may vary based on where it is located on a garment 510. In this latter aspect, a processing resource such as the controller 530 or some other local or remote processing resource coupled to the module 520 may detect the location and adapt processing of data from the module 520 based on the location. This may, for example, include a selection of different models, algorithms, or parameters for processing sensed data.

In another aspect, this may include selecting from among a variety of different activity recognition models based on the detected location. For example, a variety of different activity recognition models may be developed, such as machine learning models, lookup tables, analytical models, or the like, which may be applied to accelerometer data to detect an activity type. Other motion data, such as gyroscope data, may also or instead be used, and activity recognition processes may also be augmented by other potentially relevant data, such as data from a barometer, magnetometer, GPS, and so forth. This may generally discriminate, e.g., between being asleep, at rest, or in motion, or this may discriminate more finely among different types of athletic activity, such as walking, running, biking, swimming, playing tennis, playing squash, and so forth. While useful models may be developed for detecting activities in this manner, the nature of the detection will depend upon where the accelerometers are located on a body. Thus, a processing resource may usefully identify location first using location detection systems (such as tags, electromechanical bus connections, etc.) built into the garment 510, and then use this detected location to select a suitable model for activity recognition. This technique may similarly be applied to calibration models, physiological signals processing models, and the like, or to otherwise adapt processing of signals from a module 520 based on the location of the module 520. In general, determining a location of a module 520 may include, e.g., receiving a sensed location for the module 520, determining the location based on communications between the module 520 and the garment 510, determining the location based on data received from a physiological sensor 522 of the module 520, and so forth.

Once determined using any of the techniques above, the location of a module 520 may be transmitted for storage and analysis to a remote processing facility 550, a database 560, or the like. That is, in addition to the module 520 using this information locally to configure itself for the location in which it is worn, the module 520 may communicate this information to other modules 520, peripherals, or the cloud. Processing this information in the cloud may help an organization determine if a module 520 has ever been installed on a garment 510, which locations are most used, and how modules 520 perform differently in different locations. These analytics may be useful for many purposes and may, for example, be used to improve the design or use of modules 520 and garments 510, either for a population, for a user type, or for a particular user.

As stated above, the system 500 may further include a processor 540 and a memory 542. In general, the memory 542 may bear computer-executable code configured to be executed by the processor 540 to perform processing of the data received from one or more modules 520. One or more of the processor 540 and the memory 542 may be located on a local component of the system 500 (e.g., the garment 510, a module 520, the controller 530, and the like) or as part of a remote processing facility 550 or the like, as shown in the figure. Thus, in an aspect, one or more of the processor 540 and the memory 542 are included on at least one of the plurality of modules 520. In this manner, processing may be performed on a central module or on each module 520 independently. In another aspect, one or more of the processor 540 and the memory 542 are remote relative to each of the plurality of modules 520. For example, processing may be performed on a connected peripheral device, such as a smartphone, laptop, local computer, or cloud resource.

The processor 540 may be configured to assess the quality of the data received from a physiological sensor 522 of the module 520, or otherwise process data as described herein. The memory 542 may store one or more algorithms, models, and supporting data (e.g., parameters, calibration results, user selections, and so forth) and the like for transforming data received from a physiological sensor 522 of the module 520. In this manner, suitable models, algorithms, tuning parameters, and the like may be selected for use in transforming the data based on the location of the module 520 as determined by the controller 530 and/or processor 540 as described herein.

A database 560 may be located remotely and in communication with the system 500 via the data network 502. The database 560 may store data related to the system 500 such as any discussed herein—e.g., sensed data, processed data, transformed data, metadata, physiological signal processing models and algorithms, personal activity history, and the like. The system 500 may further include one or more servers 570 that host data, provide a user interface, process data, and so forth in order to facilitate use of the modules 520 and garments 510 as described herein.

It will be appreciated that the garment 510, modules 520, and accompanying garment infrastructure and remote networking/processing resources may advantageously be used in combination to improve physiological monitoring and achieve modes of monitoring not previously available.

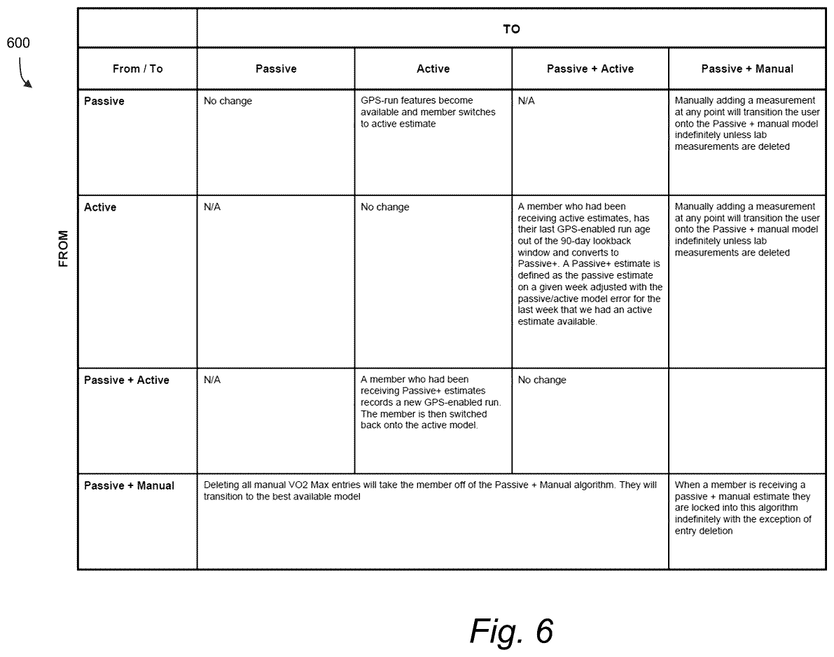

FIG. 6 illustrates a strategy for selecting among modes to calculate VO2 Max. In general, these strategies, and the systems and methods implementing them, may be used with any of the physiological monitoring systems described herein to improve estimates of VO2 Max for a user. Maximal oxygen consumption (VO2 Max) represents the maximum amount of oxygen that a person can use during intense exercise. It is a measure of aerobic fitness, or the body's ability to deliver and utilize oxygen during physical activity. Maximal oxygen consumption—typically measured as ml/kg/minute (milliliters of oxygen consumed per kilogram of body weight per minute of exercise)—can provide a key measure of cardiorespiratory fitness. Maximal oxygen consumption can be a strong indicator of overall health that is correlated to many specific health benefits, and more generally serves as a figure of merit indicating a reduced risk of all-cause mortality.

Wearable devices designed for VO2 Max monitoring may use a combination of sensors to estimate this value. These include photoplethysmography (PPG) sensors for heart rate monitoring, accelerometers and gyroscopes for motion tracking, and electrocardiogram (ECG) sensors to capture electrical activity of the heart. Some advanced wearables integrate respiratory rate monitoring and blood oxygen saturation (SpO2) sensors to improve VO2 Max estimation. A wearable device may, for example, calculate VO2 Max by analyzing heart rate data in relation to motion and exertion levels, often comparing this data against standardized fitness models.

However, accurate measurement of VO2 Max has traditionally required laboratory settings equipped with specialized equipment and a strenuous user protocol. While other portable and wearable devices have been developed that facilitate an accurate VO2 Max assessment in more accessible environments, there remains a need for simplified VO2 Max monitoring that can be performed transparently based on data from a wearable physiological monitor, e.g., without guided protocols or user attention.

As shown in FIG. 6, a strategy 600 for selecting among calculation modes uses an estimation framework with a plurality of VO2 Max estimation modes whose availability depends upon which input feature sets are presently available for a member. In one implementation, the framework includes a passive model that relies on inputs generally available from continuous wearable monitoring, and an active model that additionally incorporates GPS-enabled running features when such data is available within a predetermined recency window. The framework may further support incorporation of user-provided VO2 Max measurements (also referred to as manual entries), which may be treated as higher-confidence supplemental information that can be used to calibrate or override wearable-derived estimates. To manage transitions among these modes in a stable and predictable manner, a transition matrix may define permissible state changes and associated required inputs and may also define ranking logic to select a member-facing VO2 Max value when multiple candidate estimates exist for a given scoring event.

In general, a transition may be triggered by a periodic scoring event (e.g., a weekly update performed shortly after sleep/recovery processing), and the system may determine which of several modes are computable at that time based on available data, including a passive estimate, an active estimate, and so forth. For example, when GPS-run features become available, e.g., after a user completes a GPS-enabled run, a user previously receiving passive estimates may be transitioned to an active estimate by computing the active model output and displaying it in preference to the passive output. Conversely, when a member who has been receiving active estimates no longer has qualifying GPS-enabled run data within a lookback interval (e.g., ninety days), the member may transition to a “passive-plus” mode in which the passive estimate is adjusted using an offset derived from the most recent time at which both passive and active estimates were concurrently available. In this manner, the system may preserve continuity across the active-to-passive transition, while also propagating information gained from the active feature set forward in time after the active feature set becomes stale.

Manual entries may introduce an additional, higher-precedence pathway through the transition matrix. A member may enter a manual VO2 Max value with an associated score date, and the system may rescore historical estimates over a window spanning from the score date to the entry date subject to recency constraints (e.g., only altering member-facing scores when the score date is within a predetermined number of days of an estimated VO2 Max). Upon a manual entry, the member may transition to a “passive-plus-manual” mode, where the passive model remains the baseline estimator while a passive-to-manual mapping (e.g., an offset or other correction term derived from agreement between the manual value and a contemporaneous passive estimate) is applied to subsequent estimates. In some implementations, once a member is receiving passive-plus-manual estimates, the member remains in that mode indefinitely unless manual entries are removed, thereby ensuring that the calibrating effect of a user-provided measurement is preserved until the user explicitly withdraws it.

The transition matrix may also encode which transitions are not permitted and which intermediate computations are required to effect a permitted transition. For example, where an active estimate is not computable because recent GPS-run features are absent, the matrix may indicate that an active estimate is unavailable and that the system instead uses the passive estimate or a passive estimate adjusted by a previously established passive-to-active mapping. Similarly, when manual entries are present, the matrix may indicate that manual-related inputs (e.g., most recent manual value and manual input date) are required and that the manual-based modes supersede active modes in member-facing outputs. In operation, the system may compute multiple candidate estimates during a scoring cycle and then select the member-facing estimate according to a predetermined precedence order, such that, by way of example, an exact manual entry may be displayed immediately following entry, followed by passive-plus-manual, followed by active, followed by passive-plus-active, followed by passive, thereby favoring estimates expected to have higher accuracy while maintaining stability during changes in data availability.

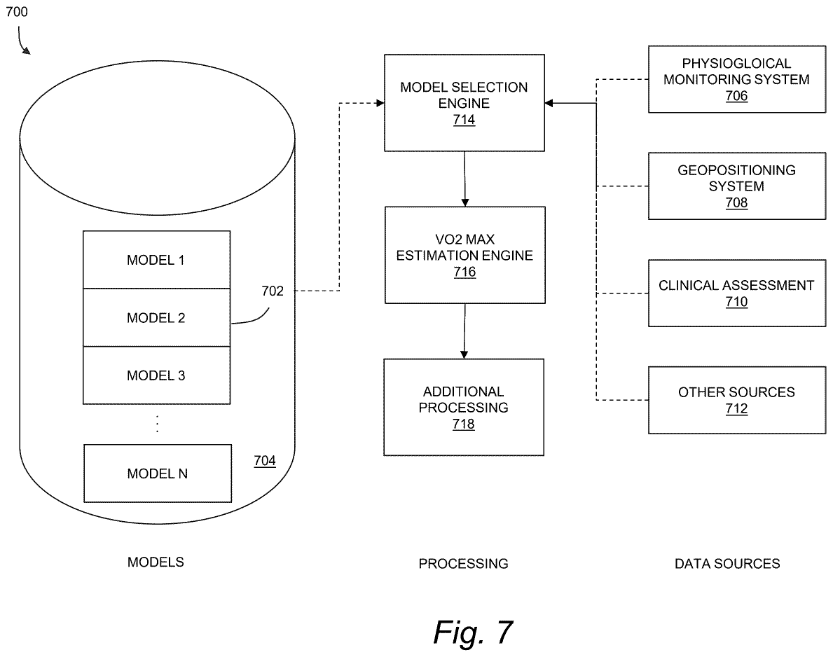

FIG. 7 illustrates a system for estimating maximal oxygen consumption for a user. In general, the system 700 may include models (including any data, weights, coefficients, and the like that are precomputed and used at inference time), data sources, and processing in a cooperative manner to support context-dependent calculations of maximal oxygen consumption. Model and data selection may use any suitable selection strategy, state machine, or the like, such as the selection strategy described with reference to FIG. 6, or any other strategy or combination of strategies suitable for selecting and applying the base model for a current user context, maintaining scoring continuity, and so forth.

A variety of models 702 (labeled “MODEL 1” through “MODEL N” in the figure) may be stored in a data store 704, such as any suitable database, data repository, or the like, where they can be accessed as needed. The models 702 may, for example, reside on a physiological monitor, a user device, a server, or any of the other computing devices described herein. Each of the models 702 may be adapted to the availability of data in one or more data sources for the user. For example, the different data sources may provide different types of data, and these types of data may support differentiated (e.g., better or worse) estimates, or these types of data may contain more or less accurate estimates or evaluations of maximal oxygen consumption. Thus, each model may support a different mode of calculating the maximal oxygen consumption that is optimized for a particular availability of data in a particular group of the data sources.

For example, a first model of the one of the models 702 may be used to estimate the maximal oxygen consumption for a user without any explicit or implicit measurements of maximal oxygen consumption. For example, a regression model or the like may be created for a population of users, a sub-population, or demographic group of users, or an individual, that relates clinically measured maximal oxygen consumption to a suite of data and statistical descriptors from a continuous wearable physiological monitor, such as any of the monitors described herein. A model may also or instead be based on data and protocols for submaximal testing known to approximate maximal oxygen consumption, using, e.g., submaximal tests, running/cycling tests using speed or power, and so forth. By way of non-limiting examples, user data, such as gender, body mass index, maximum heart rate, and age, have been shown to be related to maximal oxygen consumption. Similarly, statistics derived from physiological monitoring data may also be useful, such as sleep consistency, median or mean daily maximum heart rate, an average or other measure of daily heart rate zones (for one or more of zone 2, zone 3, zone 4, and zone 5), and so forth. While daily measures of any of the foregoing may be used, a mean, median, percentile, range, or other measure over a historical window may also or instead be used. As a significant advantage, this avoids the outsized influence of individual daily variations that may be unrelated to maximal oxygen consumption in isolation, such as poor sleep, high prior-day strain, high stress, illness, and so forth. As another advantage, using a historical window of 30, 45, 60, or 90 days, or some other window, can provide a more continuous measure of maximal oxygen consumption, and a more consistent user experience, by smoothing out daily variations in individual metrics.

Other metrics may also or instead be used to estimate maximal oxygen consumption in a first model. For example, in one aspect, descriptive statistics of heart rate zone data over the entire day may be used. In another aspect, the dynamic changes in heart rate may be used, such as the user's heart rate recovery, or the rate at which the heart rate falls when a user transitions from high intensity or exertion to a period of rest. In another aspect, changes in heart rate may be measured in relation to metrics for motion, e.g., data from accelerometers and/or gyroscopes of a wearable monitor. This approach usefully permits an evaluation of movement without requiring geolocation services. In another aspect, changes in a PPG signal may be used, e.g., where the PPG waveform encodes information about the individual's cardiovascular system (i.e., systolic peak, diastolic peak, dicrotic notch) that permits inferences about cardiovascular health or performance.

For some metrics, the more recent history may also or instead be relevant. Thus, for example, for sleep metrics, strain metrics, and the like, a shorter-duration window (e.g., 4-7 days) may also or instead be used to account more directly for the current impact of recent user experiences. In some cases, a descriptive statistic, such as maximum heart rate, may be estimated based on, e.g., age, input by the user, or evaluated based on continuously monitored heart rate data for the user. In general, these and other statistics derived from continuous monitoring may be used as regressors in a regression model, such as a linear regression model, ridge regression model, or any other suitable regression model or the like, to create a formula for calculating an estimated maximal oxygen consumption. The first model may provide a default model for use in estimating maximal oxygen consumption when other, potentially more accurate data is not available.

A second model of the one or more models may use a maximal oxygen consumption that is actively measured using a wearable device and a predetermined measurement protocol. For example, a user may be requested to run for a certain speed, distance, and/or time while actively monitoring various heart rate metrics and monitoring physical movement with GPS metrics, or a user may simply record a run with GPS tracking, and the system may evaluate whether data from the run is suitable for estimating maximal oxygen consumption using the second model. In general, the model may include any suitable regression model, and may use any of the regressors discussed above, as well as GPS data from the run, such as statistical measures of pace, distance, altitude, and so forth. In one aspect, derived statistics may be further normalized according to heart rate. For example, the average running pace divided by a percent of maximum heart rate may be calculated for GPS-enabled runs, or a percentile such as the 25^{th} percentile or 75^{th} percentile of the running pace divided by maximum heart rate, or some combination of these or similar metrics. In general, these metrics characterize the relationship between running speed and the runner's heart rate for use in estimating maximal oxygen consumption. Other factors, such as a standard deviation or range of altitudes, may also be used to account for ascent and descent when estimating maximal oxygen consumption. The second model provides an improved, e.g., more accurate, model (relative to the first model) for estimating maximal oxygen consumption when data such as GPS-enabled run data is available for the user. It will be understood that a GPS-based model may use some or all of the inputs that are used by the first model.

GPS data may also or instead permit additional inferences that improve the accuracy of exertion measurements. For example, GPS data and time/date may be used to infer the conditions of a run or bicycle ride, such as terrain, climate, altitude above sea level, temperature, weather (e.g., precipitation, frozen terrain, etc.), and so forth, in order to account for how these conditions might affect cardiovascular performance and the load or exertion required by the exercise.

In one aspect, the higher accuracy model based on GPS-enabled run data may be used to calculate a one-off estimate of maximal oxygen consumption that can be used to adjust subsequent calculations using the first model. That is, a difference may be determined between the estimate calculated with the first model and the estimate calculated using the GPS-enabled run data, and that difference may be used to adjust estimates calculated with the first model for some predetermined amount of time going forward. Because maximal oxygen consumption can change over time, this difference-based adjustment may be phased out over some period of time, or removed after some interval, such as one week, one month, two months, and so forth, and the system 700 may return to the first model for subsequent calculations.

A third model may use the results from a clinical assessment of maximal oxygen consumption. In general, the clinical protocol for measuring VO₂ max (maximal oxygen consumption) involves a graded exercise test (GXT) performed in a controlled laboratory setting, typically using a treadmill or cycle ergometer. The test is designed to progressively increase exercise intensity while measuring oxygen consumption (VO₂), carbon dioxide production (VCO₂), and other physiological responses. A variety of more specific protocols are known in the art, such as the Bruce Treadmill Protocol (speed and incline increase every 3 minutes), the Modified Balke Protocol (gradual incline increases with constant speed), and the Cycle Ergometer Protocol (resistance increases in set increments, e.g., 15-25 watts per minute). A variety of termination criteria may also be used. For example, the test may continue until the participant reaches volitional exhaustion, or until one or more physiological termination criteria occur (e.g., ECG abnormalities, excessive blood pressure rise, or respiratory exchange ratio (RER)>1.1). The VO₂ max is then identified where oxygen consumption plateaus despite increased workload, the respiratory exchange ratio (RER) exceeds 1.1, the heart rate reaches 85-100% of age-predicted maximum, the blood lactate concentration rises significantly (if measured), and/or the participant reaches volitional fatigue. For populations unable to perform a maximal test, submaximal tests (e.g., Astrand-Rhyming Cycle Test, Rockport Walk Test, or YMCA Step Test) can estimate VO₂ max using heart rate response to exercise.

It will be noted that the second model and the third model may use different exit conditions. For example, the second model (GPS-enabled run data) may be phased out over time, while the third model may be used indefinitely when clinical assessment data becomes available. While it is possible to treat these two supplemental data sources the same, e.g., both phasing out over time or both lasting indefinitely, they may also be treated differently, where, for example, the clinical assessment is assumed to be a more accurate, ground truth measurement of maximal oxygen consumption that is suitable for use as a benchmark until conflicting or more recent data becomes available.

Regardless of which protocol is used, once a clinical assessment of maximal oxygen consumption is available, this may be used to improve the accuracy of estimates calculated using other techniques. For example, in one aspect, a difference may be determined between the clinical assessment value and an estimate that was calculated using, e.g., the first model (as applied to data that was acquired on the same day as the clinical assessment). This difference may then be used to prospectively adjust subsequent calculations of maximal oxygen consumption with the first model, and may continue indefinitely. Use of the third model may be terminated under various conditions, e.g., if the user-provided clinical assessment is deleted, or if a newer, more recent clinical assessment is provided. The use of the third model, or any other model that is dependent on additional data sources, may also or instead be aged out, either in a step function or a gradual transition, using any suitable aging rate and interval based on, e.g., the reliability of the additional data source(s), the rate at which VO2 Max is observed to change for an individual or a population, or using any other criteria or metrics.

Other models may also or instead be used, including, e.g., models for different user contexts (e.g., poor sleep, high strain, etc.), or models based on availability of other data that might improve the accuracy of estimates, such as continuous GPS data, hydration data, user workout descriptions, a data feed from a VO2 monitor intermittently or continuously worn by the user, and so forth. Furthermore, while linear regression techniques are described, the first, second, and third models may also or instead use a variety of other machine learning techniques that may be used to estimate VO2 Max for a data set, including, without limitation, regression, polynomial regression, and decision tree regression, which are typically used to predict continuous variables, as well as neural networks, including deep learning architectures, which are often effective for complex, non-linear estimation tasks, and clustering methods such as k-means and hierarchical clustering, which can assist in estimating distributions and identifying patterns in data.

In one aspect, the difference used to adjust a base model (e.g., the first model) according to a measurement of VO2 Max (e.g., a GPS-based run or a clinical assessment) may itself use an offset that adapts to user context. That is, rather than a static difference based on a single comparison, an amount of the difference may change over time based on a learned offset pattern for the user, e.g., based on changes to physiology, current conditions, etc. For example, a historically highly fit athlete who has taken several months off of exercise, e.g., due to an injury or personal circumstances, may still have a high VO2 Max. However, a system that follows the base model by adjusting this initial, high reading upward as the subject's lifestyle improves may reach an unrealistically or unachievably high estimated VO2 Max, rather than tapering as a ceiling for the subject is reached. More generally, the offset may change as a user's VO2 Max increases toward a ceiling or decreases toward a floor over time, and the difference-based adjustment may usefully account for these types of changes, e.g., by training an offset model to learn how to adjust the base model output as physiology changes over time.

In another aspect, one or more of the models may synthesize multiple models or other estimator outputs, or select from among different calculation methods based on user context. For example, a model may usefully synthesize estimates of VO2 Max from different models such as a pulse-level model that performs instantaneous estimates based on raw photoplethysmography data, an event-level model that encodes one second intervals over the course of a workout or other activity, and a long-term model that encodes general behavior based on daily or weekly behavioral metrics.

More generally, any technique that can be trained to estimate a continuous variable based on a labeled data set may be used to derive a model for VO2 Max estimation as described herein. In one aspect, the first model may be the most accurate model that can be derived using only data that can be obtained from a wearable monitor. Depending on the wearable monitor, this may include heart rate data, motion data, GPS data, temperature data, and so forth. Thus, in one aspect, the choice of the first model may depend on the particular wearable monitor being used and the best (e.g., most accurate) model available for that data set. In another aspect, there is disclosed herein an approach to VO2 Max estimation that contemplates numerous models created for various user data contexts, along with rules for switching between the available models according to the current data sources.

The system 700 may include a variety of data sources useful for estimating maximal oxygen consumption. Each data source may have different data availability, so that the selection of a particular model will depend on which of the data sources are presently available for use in calculating estimates.

The data sources may, for example, include a physiological monitoring system 706, such as any of the physiological monitoring systems 706 described herein. In one aspect, raw physiological data may be provided, e.g., by a wearable monitor, such as PPG data, motion data, GPS data, or the like from a wearable monitor. In general, the available data may include heart rate data, sleep data, heart rate variability data, motion data, skin temperature data, and so forth. Other derived data, such as respiration rate or blood pressure, may also or instead be made available for estimates where the data is available from the physiological monitoring system 706, or can be derived from such data, and can be correlated to maximal oxygen consumption in a way that supports any of the models described herein.

In some embodiments, a VO2 Max estimation model may be trained and/or executed using a feature set derived from continuous wearable monitoring and, when available, supplemental activity data. By way of example and not limitation, model features may include demographic and profile features such as age, sex, or a physiological-baseline category, height, weight, body mass index, and an estimated or user-specified maximum heart rate. Model features may also include descriptive statistics computed over an historical lookback window (e.g., about 30, 60, or 90 days) from physiological and activity signals including resting heart rate measured during sleep, heart rate variability, day-average heart rate, day-maximum heart rate, day strain or other exertion scores, and sleep metrics such as sleep consistency. Time-in-zone features may also be used, such as average, median, percentile, or total time spent in one or more heart rate zones (e.g., zones 2-5, including combined zone 4/5 time), optionally including transformed versions of these features (e.g., log-scaled zone-time values) to improve model fit and robustness.

The data sources may also or instead include a Global Positioning System (GPS) 708. In one aspect, the physiological monitoring system 706 may include the GPS 708, and may provide any corresponding location data. In another aspect, a supplemental user device such as a smartphone or other wearable or portable electronic device may be used to acquire GPS data that can be shared with the system 700 for use in estimating maximal oxygen consumption. Where GPS-enabled activity data is available, additional features may include pace, distance, duration, and altitude/elevation statistics, and normalized exertion measures such as pace divided by a percent of maximum heart rate (e.g., a mean or percentile of pace-over-heart-rate-percent across one or more qualifying runs), thereby characterizing cardiovascular response relative to external workload for estimating VO2 Max.

The data sources may also or instead include a clinical assessment 710 of maximal oxygen consumption. This may include any corresponding test or assessment by the user, and in particular, tests occurring on or near a day for which data is available from the physiological monitoring system 706. This may include a clinical assessment using, e.g., any of the techniques or protocols described herein, and may be reported to the system 700 by the user, by a physician for the user, or by any other healthcare professional or the like that conducted the assessment.

The data sources may also or instead include any other sources 712 of data useful for obtaining more accurate estimates of maximal oxygen consumption. This may, for example, include other monitoring devices such as portable VO2 Max monitors, perspiration monitors, smart scales (for weight or body fat), oxygen saturation monitors, erg or other physical output monitors, and so forth. This may also or instead include other self-reporting sources such as user-measured or user-calculated metrics for weight, body mass index, VO2 Max, resting heart rate, maximum heart rate, and so forth. In another aspect, a treadmill, stair climber, elliptical, rowing machine, or the like may be used to measure physical output by a user while acquiring data from the physiological monitoring system 706. This approach can provide a useful alternative to GPS-enabled run data that includes a measure of physical output and concurrent user physiological response without requiring location tracking. More generally, any other data sources that can be correlated to maximal oxygen consumption in order to support improved modeling and estimation may be used as data sources to support the system 700 described herein.

The system 700 may include a number of processing engines or computing modules that implement a method as described herein, e.g., for selecting among different modes for calculating VO2 Max based on available data. These models may be realized, e.g., as computer-executable code stored in a memory and executable by one or more processors to perform various supporting tasks and functions.

The processing may include a model selection engine 714. In general, the model selection engine 714 may evaluate the one or more data sources to identify data sources that are presently available and may select one of the models 702 based on the availability of data in the one or more presently available data sources. This evaluation may be performed at any suitable interval. For example, where a maximal oxygen consumption is reported to a user daily, the model selection engine 714 may determine the availability of data prior to the daily estimation, and then select a suitable one of the models 702 based on the availability of data for that reporting day. It will be appreciated that availability need not be instantaneously evaluated. That is, if a new data source becomes available or becomes unavailable, that availability will be buffered for use when the next model selection is required. It will also be understood that availability may be windowed. For example, a clinical assessment of maximal oxygen consumption may be useful for one or two days before and after the date of the assessment. Thus, in that context, availability may mean availability within a two-day window around the estimation date.

It will also be understood that a variety of transition rules may be useful for determining when to transition from one model to another model. For example, as noted above, clinical assessments may remain significant for some interval, such as several days or several weeks, but they are not perpetually accurate, and might be presumed to be the most accurate on the day that they are taken. Thus, a rule may be provided that a clinical assessment may be used with the third model described above (e.g., based on modifying a calculation from the first model based on the clinical assessment) only in those cases where data for the first model is available from the physiological monitoring system 706 on the date of the clinical assessment, or within one or two days before or after the date of the clinical assessment. Of course, a more or less permissive rule may also be used. For example, if maximal oxygen consumption is viewed as fairly stable over time, the system 700 may transition to the third model if data is available from the physiological monitoring system 706 within seven or more days of the clinical assessment. In another aspect, a more restrictive model may be used such that the third model is only used when data from the physiological monitoring system 706 is available on the same day as the clinical assessment.

In another aspect, once the third model—a calculation with the first model that is corrected based on a clinical assessment—is selected for a user, that model may continue to be used indefinitely. A variety of exit conditions for use of the third model may also be provided. For example, if the clinical assessment 710 is deleted from the system 700 or otherwise flagged by the user for non-use, the model selection engine 714 may either (a) return to a prior clinical assessment, if available, or (b) change to a next best model, which may be any other one of the estimation models 702 described herein. The order of priority for selecting a next best model may be predetermined according to, e.g., expected accuracy or any other suitable criteria.

In another aspect, where GPS-enabled run data is available, e.g., GPS data acquired concurrently with data from the physiological monitoring system 706 during strenuous exercise such as a run, the second model described above may be used for estimating maximal oxygen consumption. In one aspect, this model may have an intermediate priority so that it is replaced by the third model when clinical assessment data is available. In another aspect, where GPS-enabled run data becomes available, inference may be performed using two or more estimation models (e.g., the first model and the second model), and a final estimate may be calculated using a combination of model outputs and available contextual data. Thus, in one aspect, the second model may be used in combination with the first model to provide an improved estimate when, e.g., GPS-enabled run data (or other objective measurements of physical output) is available. The second model may, for example, only be conditionally used where there is suitable supporting data, such as at least one GPS-enabled run in the last ninety days. In this case, prior to transitioning back to the first model after the expiration of GPS-enabled run data, a most recent measurement using the second model (e.g., that accounts for active data such as one or more GPS-enabled runs) can still be used prospectively to adjust the first model until additional inputs are available. In another aspect, the system 700 may generally monitor agreement between models, e.g., between the first model and the second model, and may smooth transitions between the first model and the second model to maintain stability of estimates as the user context changes periodically between a usable history of GPS-enabled runs or similar activity, and the absence of GPS-enabled run data.

In another aspect, the first model, e.g., a regression model or other model that estimates maximal oxygen consumption based on a suite of data and/or derived metrics from the physiological monitoring system 706, may be used as a default model where other sources of data are unavailable. This provides a baseline estimate, with the possibility of moving to more accurate models when additional data becomes available.

Once a model selection has been made, a VO2 Max estimation engine 716 may be used to calculate a maximal oxygen consumption for the user based on the selected model.

Additional processing 718 may also be provided. This may, for example, include storing the estimated maximal oxygen consumption, displaying the estimated maximal oxygen consumption to a user, providing coaching or feedback to the user based on the estimated maximal oxygen consumption, tracking changes to maximal oxygen consumption over time, and so forth.

According to the foregoing, in one aspect a system disclosed herein includes: a physiological monitor configured to continuously acquire heart rate data from a user; a memory storing a plurality of models for estimating maximal oxygen consumption for the user, each of the models adapted to an availability of data in one or more data sources for the user; and a computing device configured by computer-executable code to estimate the maximal oxygen consumption for the user by: receiving data from at least one of the one or more data sources for the user, selecting one of the plurality of models based on the availability of data in the at least one of the one or more data sources, and estimating the maximal oxygen consumption for the user based on the selected model and the user data.

The computing device may be configured to transmit the maximal oxygen consumption to a user device for display. The computing device may, for example, be a wearable physiological monitor, a user device associated with a user of a wearable physiological monitor, a server supporting a physiological monitoring system and coupled in a communicating relationship with a wearable physiological monitor, or some combination of these.

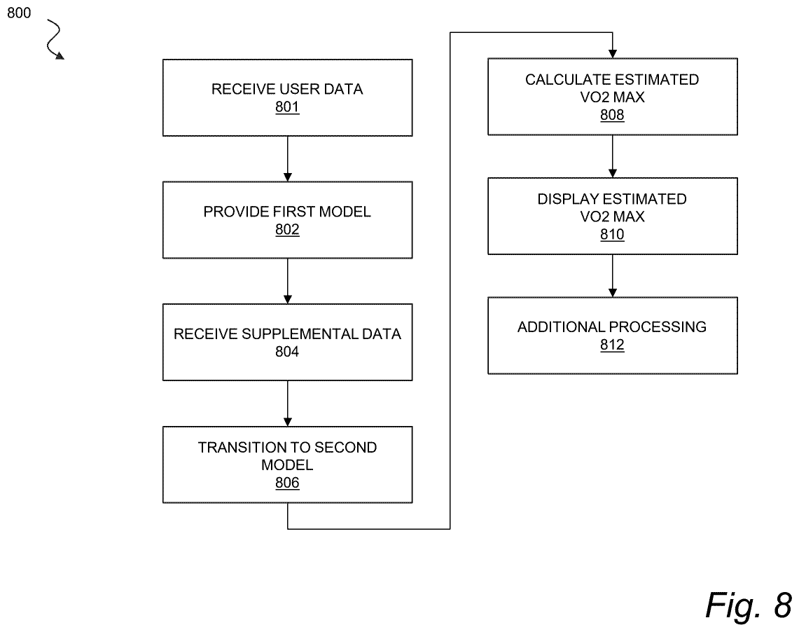

FIG. 8 is a flow chart of a method for estimating maximal oxygen consumption. In general, the method 800 described herein includes providing a number of models for estimating maximal oxygen consumption and selecting among the models based on available data. As a significant technical advantage, this approach supports adjusting model selections to meet changing user context, and more specifically the use of progressively higher quality models when supported by data availability.

As shown in step 801, the method 800 may include receiving user data, such as any data from any of the data sources described above. For example, the user data may include photoplethysmography data received from a user. In one aspect, receiving the user data may include detecting sleep based on the user data, and then using the detected sleep to identify sleep patterns, as well as to identify periods within sleep intervals where a representative measurement of cardiac activity (such as heart rate or heart rate variability) can be taken. Thus, in one aspect, an incoming stream of photoplethysmography data may be received and analyzed to identify sleep intervals, after which suitable periods of sleep may be identified for further processing. The user data may also or instead include demographic data for the user, such as gender and age. In another aspect, the user data may include one or more descriptive statistics for heart rate zones of the user during activity derived from the physiological data. Heart rate zone data may be based on, e.g., data acquired by a continuous physiological monitor worn by the user or other suitable source(s) of heart rate data.

As shown in step 802, the method 800 may include providing a first model for estimating a maximal oxygen consumption for a user based on user data, the user data including physiological data acquired from a wearable physiological monitor worn by the user. This may, for example, include selecting or storing any of the models described herein, such as a regression model and/or adjustments thereto. The first model may include a linear regression model or any other regression model or other model and/or adjustments thereto, as generally described herein.

As shown in step 804, the method 800 may include receiving supplemental data relating to the maximal oxygen consumption. This may, for example, include a clinical assessment of maximal oxygen consumption, data for a GPS-enabled run, or any of the other supplemental data described herein. In one aspect, a user may provide data, such as a clinical assessment of VO2 Max, at any time, and with any measurement date. If there is corresponding VO2 Max estimate data from some other model, then this clinical assessment may be used to update all measurements after the effective date of the measurement, even if the measurement was several months in the past. This may be particularly useful where a user begins VO2 Max measurements some significant amount of time after the user begins monitoring other metrics with a wearable device.

As shown in step 806, the method may include transitioning to a second model for estimating the maximal oxygen consumption based on the user data and the supplemental data. In general, this may include transitioning to any of the other models described herein, e.g., based on transition rules or logic for selecting between models based on data availability. This may also or instead include transitioning to a combination of models, e.g., when GPS run data becomes available, as described above, or other data for exertion under known loads becomes available that can be used for improved VO2 Max estimation based on physical activity.

Thus, in one aspect, the transition may include a transition from the first model to a hybrid estimation using the first model and the second model. In another aspect, the transition may include a transition from any combination of models described herein to a model that includes a correction based on a clinical assessment of VO2 Max, e.g., the third model described above. In another aspect, this may include transitioning away from the third model to any of the other models or combination of models, e.g., when a clinical assessment of VO2 Max is deleted from the data by a user. More generally, any transition between one or more of the models, or combinations of models, may be triggered according to a suitable transition rule based on the availability (or age) of data, such as any of the transition rules described herein.

As shown in step 808, the method 800 may include calculating an estimated maximal oxygen consumption based on the second model. The second model may, for example, include any of the linear regression models and/or adjustments thereto as described herein. In one aspect, the supplemental data may include Global Positioning System (GPS) data acquired for the user during a run, and the second model may estimate the maximal oxygen consumption based in part on a portion of the user data acquired during the run and the GPS data acquired during the run, or as described above, based on a combination of a GPS-based model and a non-GPS-based model, such as the first and second models described above. It will be appreciated that, while a GPS-enabled run provides good data for refining the accuracy of a VO2 Max calculation for a user, other data sources and types may also or instead be used to refine a VO2 Max measurement. For example, even without tracking a specific run, where GPS data is generally available for a user, this may be used to evaluate physical output and refine VO2 Max estimation. Similarly, where a user exercises under other measurable conditions (e.g., measuring physical output with an erg machine, or measuring a proxy for physical output such as angle and speed for a treadmill, or speed+weight for a stair machine), may also or instead be used. Similarly, motion data from a wearable device such as data from accelerometers and gyroscopes may also or instead be used to estimate physical output, and to support refined VO2 Max calculations as described herein. In one aspect, different models may be provided for two or more of these different techniques for measuring physical output, and the results may be synthesized by combining the output from various models and/or intermittently selecting different models based on the nature of data for a time window, or any suitable combination of these. More generally, any technique or combination of techniques for combining data about an internal physical load (e.g., heart rate) and external physical load (e.g., weight, motion, distance, rate, elevation, etc.) may be used to refine VO2 Max estimation and support model selection as contemplated herein.

In another aspect, the supplemental data includes a clinical measurement of the maximal oxygen consumption, and the second model adjusts a result of the first model according to the clinical measurement of the maximal oxygen consumption. The second model may also or instead adjust a result of the first model according to a difference between the clinical measurement of the maximal oxygen consumption and the maximal oxygen consumption calculated according to the first model on the day of the clinical measurement of the maximal oxygen consumption.

As shown in step 810, the method 800 may include displaying the estimated maximal oxygen consumption to the user, e.g., in the user interface of a computing device associated with the user.

As shown in step 812, the method 800 may include additional processing. This may generally include any of the additional processing described herein, including storing the estimated maximal oxygen consumption, providing user feedback or coaching based on the maximal oxygen consumption, and so forth. In one aspect, additional processing may include calculating the maximal oxygen consumption once per day based on an historical window of data for the user, or otherwise repeating estimates on any suitable schedule. In another aspect, additional processing may include transitioning back to the first model based on predetermined criteria. For example, where the first model is the base or default model for estimating maximal oxygen consumption based on data from a physiological monitor, and where the supplemental data includes GPS data and/or a VO2 Max adjustment based on the GPS data, the model may transition back to the first model (or base model) after a certain period of time, e.g., one week or one month after the GPS data was acquired.

The additional processing 718 may also or instead include providing a useful user experience through a combination of data acquisition, back-end or server-side processing, and presentation through an interactive user interface of data and interactive communications. For example, in some embodiments, the systems and methods described herein provide a user-facing product experience in which a wearable physiological monitor delivers ongoing VO2 Max estimates with little or no required user attention. In general, once a sufficiency criterion for baseline data is satisfied (e.g., a threshold number of sleep sessions recorded within a preceding time window), the system may unlock VO2 Max estimation for the user and begin publishing periodic VO2 Max updates on a recurring schedule. In one implementation, a scoring event is triggered in association with recovery processing such that VO2 Max is updated shortly after sleep and recovery are scored, and the resulting VO2 Max value is stored and presented in an application user interface as a single user-facing estimate even though, internally, the system may compute multiple candidate estimates using different estimation modes.

During each scoring event, the system may automatically select among a plurality of estimation modes based on data that is available at that time, such as continuous wearable-derived physiological features, optional GPS-enabled activity features, and/or user-provided VO2 Max measurements. Where higher-quality supplemental inputs are available (e.g., recent GPS-enabled run data), the system may preferentially use a corresponding model to improve accuracy while maintaining continuity through data-driven transition logic (e.g., applying an offset between an active estimate and a passive estimate when recent GPS activity becomes stale). The system may also support manual entry of VO2 Max values with an associated score date, and, upon receiving a manual entry, may rescore one or more historical estimates and apply a mapping so that subsequent user-facing estimates remain calibrated to the manual value until the manual entry is removed. In this manner, the user can receive a stable, periodically updated VO2 Max metric that improves opportunistically when additional data becomes available, preserves continuity during changes in data availability, and permits user-supplied measurements to calibrate or override wearable-derived estimates when desired.

The systems and methods herein provide numerous improvements and technical advantages over existing VO2 Max estimation devices. For example, the techniques described herein improve the function of a wearable physiological monitoring system by dynamically selecting among multiple VO2 Max estimation modes based on objective, machine-detectable data availability conditions (e.g., presence/absence of qualifying GPS-run features within a defined recency window, and presence/absence of user-entered clinical values). This converts what would otherwise be a single static estimator into a stateful, data-driven control scheme that adapts computation to the current sensor and context signals, thereby improving the reliability of the computed VO2 Max value for real-world, continuous monitoring conditions and changing availability of external signals such as GPS.

In another aspect, the system may employ continuity-preserving mode transition logic that reduces estimation discontinuities caused by changing sensor inputs over time. In particular, when higher-information inputs (e.g., GPS-run features) become stale, the system can compute and store a mapping term (e.g., an offset derived from a most recent time when both passive and active estimates are concurrently computable) and then apply that mapping term prospectively to subsequent passive estimates. This reduces abrupt step changes in the user-facing metric that would otherwise be introduced by switching estimators, and it does so using a concrete, repeatable computation tied to sensor-derived features and a defined lookback window, improving the technical quality and stability of the output signal over time.

In another aspect, the system may advantageously employ resource-aware computation for constrained wearable ecosystems, achieved by gating higher-computational-cost feature generation and model inference on eligibility checks (e.g., only computing active-model aggregates when a qualifying run exists within a recency window; only triggering rescoring over a bounded historical window on manual entry events). By constraining when particular feature sets are computed and when rescoring is performed, the system can reduce processor cycles, memory bandwidth, storage writes, and network transmission associated with unnecessarily recomputing VO2 Max, which is a practical improvement in the operation of the overall distributed sensing system.

More generally, the systems and methods herein support deterministic reconciliation of multiple candidate estimates into a single member-facing VO2 Max value through a predefined precedence order and event-driven rescoring rules (e.g., displaying an exact manual entry immediately following entry; otherwise applying a passive-to-manual mapping; otherwise preferring active over passive-plus-active over passive). This yields predictable system behavior and provides a mechanism for coordinating heterogeneous sensor inputs and human-supplied measurements within a single physiological monitoring pipeline.

The above systems, devices, methods, processes, and the like may be realized in hardware, software, or any combination of these suitable for the control, data acquisition, and data processing described herein. This includes realization in one or more microprocessors, microcontrollers, embedded microcontrollers, programmable digital signal processors or other programmable devices or processing circuitry, along with internal and/or external memory. This may also, or instead, include one or more application-specific integrated circuits, programmable gate arrays, programmable array logic components, or any other device or devices that may be configured to process electronic signals. It will further be appreciated that a realization of the processes or devices described above may include computer-executable code created using a structured programming language such as C, an object-oriented programming language such as C++, or any other high-level or low-level programming language (including assembly languages, hardware description languages, and database programming languages and technologies) that may be stored, compiled, or interpreted to run on one of the above devices, as well as heterogeneous combinations of processors, processor architectures, or combinations of different hardware and software.

Thus, in one aspect, each method described above, and combinations thereof, may be embodied in computer-executable code that, when executing on one or more computing devices, performs the steps thereof. In another aspect, the methods may be embodied in systems that perform the steps thereof and may be distributed across devices in a number of ways, or all of the functionalities may be integrated into a dedicated, standalone device or other hardware. The code may be stored in a non-transitory fashion in a computer memory, which may be a memory from which the program executes (such as random access memory associated with a processor), or a storage device such as a disk drive, flash memory, or any other optical, electromagnetic, magnetic, infrared, or other device or combination of devices. In another aspect, any of the systems and methods described above may be embodied in any suitable transmission or propagation medium carrying computer-executable code and/or any inputs or outputs from same. In another aspect, means for performing the steps associated with the processes described above may include any of the hardware and/or software described above. All such permutations and combinations are intended to fall within the scope of the present disclosure.

The method steps of the implementations described herein are intended to include any suitable method of causing such method steps to be performed, consistent with the patentability of the following claims, unless a different meaning is expressly provided or otherwise clear from the context. So, for example, performing the step of X includes any suitable method for causing another party, such as a remote user, a remote processing resource (e.g., a server or cloud computer), or a machine, to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity and need not be located within a particular jurisdiction.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims.

What is claimed is:

1. A method comprising:
providing a first model for estimating a maximal oxygen consumption for a user based on user data, the user data including physiological data acquired from a wearable physiological monitor worn by the user;
receiving supplemental data relating to the maximal oxygen consumption;
transitioning to a second model for estimating the maximal oxygen consumption based on the user data and the supplemental data; and
calculating an estimated maximal oxygen consumption based on the second model.

2. The method of claim 1, further comprising displaying the estimated maximal oxygen consumption to the user.

3. The method of claim 1, further comprising calculating the maximal oxygen consumption once per week based on an historical window of data for the user.

4. The method of claim 1, further comprising calculating the maximal oxygen consumption once per day based on an historical window of data for the user.

5. The method of claim 1, wherein the first model includes a regression model.

6. The method of claim 1, wherein the user data includes photoplethysmography data acquired during sleep.

7. The method of claim 6, further comprising detecting the sleep based on the photoplethysmography data.

8. The method of claim 1, wherein the user data includes demographic data for the user.

9. The method of claim 1, wherein the user data includes one or more descriptive statistics for heart rate zones of the user during activity derived from the physiological data.

10. The method of claim 1, wherein the supplemental data includes Global Positioning System data acquired for the user during a run.

11. The method of claim 10, wherein the second model estimates the maximal oxygen consumption based in part on a portion of the user data acquired during the run and the Global Positioning System data acquired during the run.

12. The method of claim 11, wherein the second model includes a second regression model.

13. The method of claim 11, further comprising transitioning to the first model when the supplemental data reaches a predetermined age.

14. The method of claim 13, wherein transitioning to the first model includes using the first model with an adjustment based on an historical estimate obtained using the second model.

15. The method of claim 1, wherein the supplemental data includes a clinical measurement of the maximal oxygen consumption.

16. The method of claim 15, wherein the second model adjusts a result of the first model according to the clinical measurement of the maximal oxygen consumption.

17. The method of claim 15, wherein the second model adjusts a result of the first model according to a difference between the clinical measurement of the maximal oxygen consumption and the maximal oxygen consumption calculated according to the first model within one week of the clinical measurement of the maximal oxygen consumption.

18. A computer program product comprising computer-executable code embodied in a non-transitory computer-readable medium that, when executing on one or more computing devices, causes the one or more computing devices to perform the steps of:
receiving user data from one or more data sources for a user;
providing a plurality of models for estimating a maximal oxygen consumption for the user, each of the plurality of models adapted to an availability of data in at least one of the one or more data sources;
selecting one of the plurality of models based on the availability of data in the one or more data sources; and
estimating the maximal oxygen consumption for the user based on a selected one of the plurality of models.

19. The computer program product of claim 18, wherein the user data includes physiological data from a wearable monitor providing the physiological data while worn by the user.

20. The computer program product of claim 18, wherein the user data includes GPS data for the user acquired during a run by the user.

21. The computer program product of claim 18, wherein the user data includes a clinical measurement of the maximal oxygen consumption.

22. The computer program product of claim 18, wherein the one or more data sources include a wearable physiological monitor, a GPS device, and a clinical evaluation.

23. A system comprising:
a physiological monitor configured to continuously acquire heart rate data from a user;
a memory storing a plurality of models for estimating maximal oxygen consumption for the user, each of the plurality of models adapted to an availability of data in one or more data sources for the user; and
a computing device configured by computer-executable code to estimate the maximal oxygen consumption for the user by:
receiving user data from at least one of the one or more data sources for the user,
selecting one of the plurality of models based on the availability of data in the at least one of the one or more data sources, and
estimating the maximal oxygen consumption for the user based on a selected one of the plurality of models and the user data.

24. The system of claim 23, wherein the computing device is further configured to transmit the maximal oxygen consumption to a user device for display.
